(19) United States
(12) Patent Application Publication
(10) Pub. No.: US 2026/0209185 A1
(43) Pub. Date: Jul. 23, 2026

(54) SYNTHESIS METHODS AND INTERMEDIATES FOR THE PRODUCTION OF REMIMBRUTINIB

(71) Applicant: NOVARTIS AG, Basel (CH)

(72) Inventors: Aude FIGUCCIA, Binningen (CH); Lucie LOVELLE, Basel (CH); Michael PARMENTIER, Waldighoffen (FR)

(21) Appl. No.: 19/113,162

(22) PCT Filed: Sep. 28, 2023
(86) PCT No.: PCT/IB2023/059664

§ 371 Date: Mar. 19, 2025

Related U.S. Application Data
(60) Provisional application No. 63/377,853, filed on Sep. 30, 2022.

(51) Int. Cl. C07D 239/47; B01J 31/18; B01J 31/24; C07C 231/02; C07C 233/66

(52) U.S. Cl. CPC C07D 239/47; B01J 31/189; B01J 31/2404; C07C 231/02; C07C 233/66; B01J 2231/4211; B01J 2531/004; B01J 2531/824

(57) ABSTRACT

This invention relates to novel processes for synthesizing N-(3-(6-Amino-5-(2-(N-methylacrylamido)ethoxy)pyrimidin-4-yl)-5-fluoro-2-methylphenyl)-4-cyclopropyl-2-fluorobenzamide and to intermediates which are used in such processes.

### FIELD OF INVENTION

The present invention provides new synthetic routes, new chemical reactions, and new synthetic intermediates useful in the preparation of N-(3-(6-Amino-5-(2-(N-methylacrylamido)ethoxy)pyrimidin-4-yl)-5-fluoro-2-methylphenyl)-4-cyclopropyl-2-fluorobenzamide.

### BACKGROUND

N-(3-(6-Amino-5-(2-(N-methylacrylamido)ethoxy)pyrimidin-4-yl)-5-fluoro-2-methylphenyl)-4-cyclopropyl-2-fluorobenzamide, also known as remibrutinib, is a highly potent and selective oral Bruton's tyrosine kinase (BTK) inhibitor:

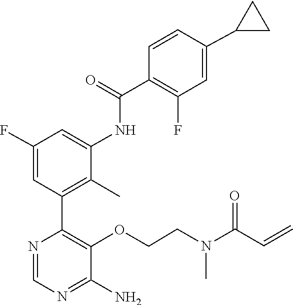 [SMILES: C=CC(=O)N(C)CCOc1c(N)ncnc1-c1cc(F)cc(NC(=O)c2ccc(C3CC3)cc2F)c1C]

Remibrutinib was first disclosed in WO2015/079417, filed Nov. 28, 2014, in Example 6. WO2015/079417 is incorporated by reference in its entirety. In WO2015/079417, Example 6(2), remibrutinib is prepared by cross-coupling of “INT 5” with “INT 8”, to give “INT 9”:

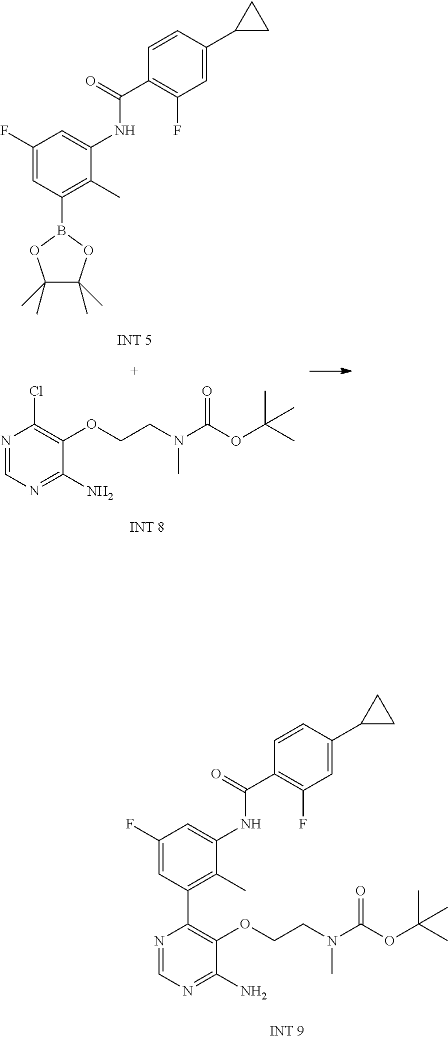 [SMILES: Cc1c(NC(=O)c2ccc(C3CC3)cc2F)cc(F)cc1B1OC(C)(C)C(C)(C)O1 ; CN(CCOc1c(N)ncnc1Cl)C(=O)OC(C)(C)C ; Cc1c(NC(=O)c2ccc(C3CC3)cc2F)cc(F)cc1-c1ncnc(N)c1OCCN(C)C(=O)OC(C)(C)C]

INT 9 is then deprotected with TFA (Example 6(3)), reacted with acrylic acid, and purified to give remibrutinib (Example 6(4)). INT 5 is a key intermediate in this process, making up one half of the structure of the final product, remibrutinib. The preparation of INT 5 is described in Example 1(5) of WO2015/079417: INT 5 is prepared by the amide coupling of INT 3 and INT 4:

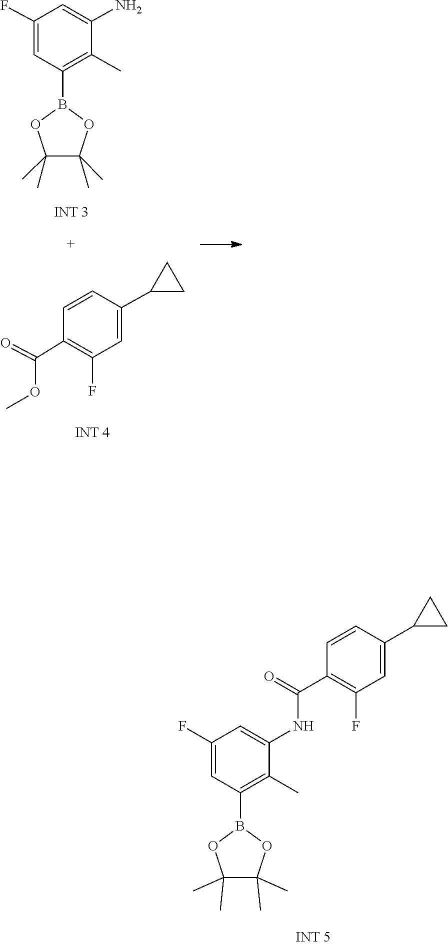 [SMILES: Cc1c(N)cc(F)cc1B1OC(C)(C)C(C)(C)O1 ; COC(=O)c1ccc(C2CC2)cc1F ; Cc1c(NC(=O)c2ccc(C3CC3)cc2F)cc(F)cc1B1OC(C)(C)C(C)(C)O1]

However, it has been discovered that INT 3 (5-fluoro-2-methyl-3-(4,4,5,5-tetramethyl-1,3,2-dioxaborolan-2-yl)aniline), is a compound with mutagenic potential and is therefore an undesirable intermediate in the synthesis of a medicinal product. The genotoxicity of INT 3 is reported for the first time in the present application.

It is therefore an object of the present invention to provide a new synthetic route to remibrutinib that minimizes contact to genotoxic reagents such as INT 3. In addition, the present invention provides improved coupling conditions for the preparation of INT 9 (referred to herein as F7) with higher yield and avoiding the need to use undesirable solvents such as as DCM (carcinogenic), DME (damage fertility), DMF (damage fertility), 1,2-dichloromethane (carcinogen).

### SUMMARY OF INVENTION

The invention provides, in a first embodiment, a synthesis method comprising converting compound X6b and compound F6 into compound F7:

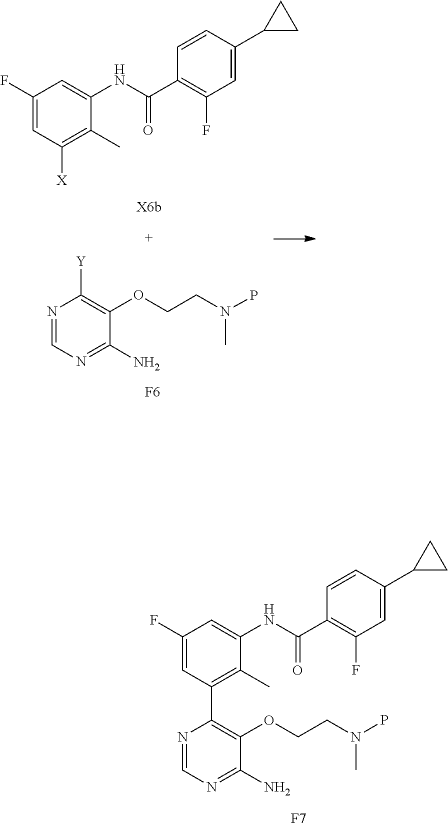

wherein X and Y are each independently F, Cl, Br, or I, and wherein P is an amine protecting group.

The invention provides, in a second embodiment, a synthesis method comprising the borylation of X6b to give X6a:

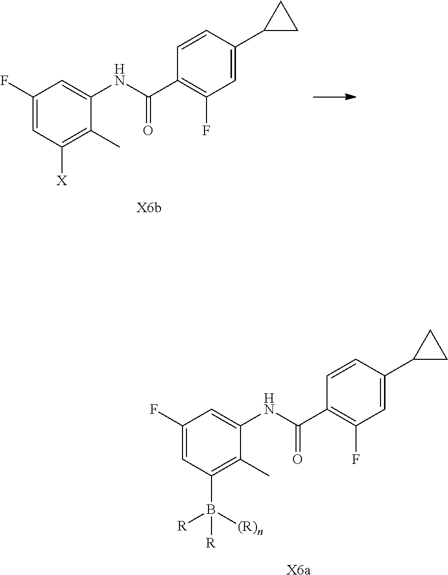

wherein X is F, Cl, Br, or I, n is 0 or 1 and R is F, Cl, Br, or I, OH, OC₁-C₆alkyl, N(C₁-C₆ alkyl)₂, aryl, or wherein two or three R groups other than F, Cl, Br, I, or OH can be taken together to form a cyclic boronate ester, for example pinacol boronate, or N-methyliminodiacetic acid (MIDA) boronate.

The invention provides, in a third embodiment, a synthetic intermediate X6b:

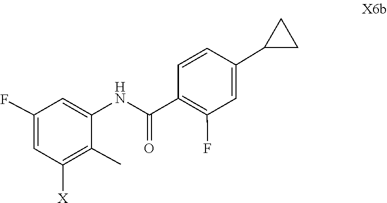

wherein X is Cl, Br, or I, preferably Br.

### DESCRIPTION OF FIGURES

FIG. 1 provides an overview of a convergent and atom efficient synthesis route for preparing remibrutinib. X6b is a key intermediate in this synthesis route.

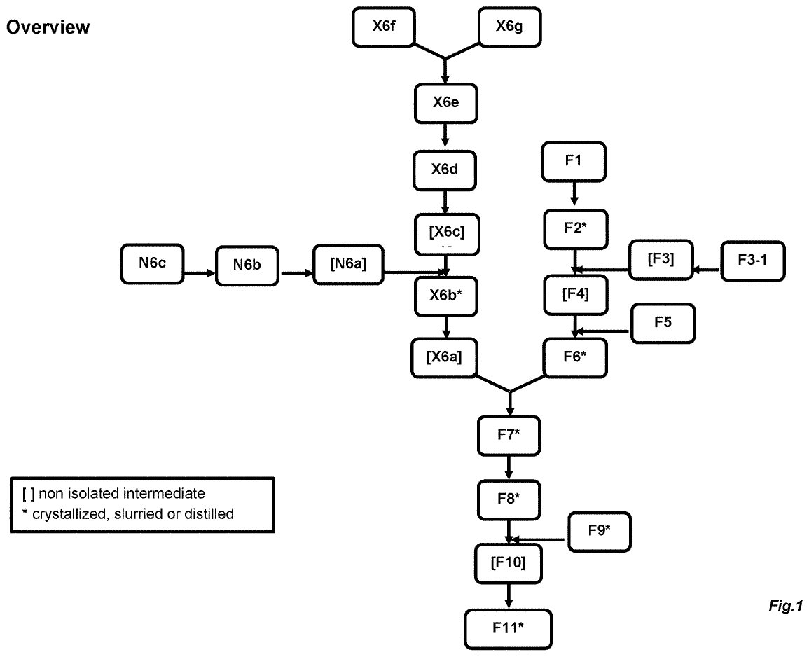

FIG. 2 demonstrates exemplary reaction conditions for the route from F1 to F6.

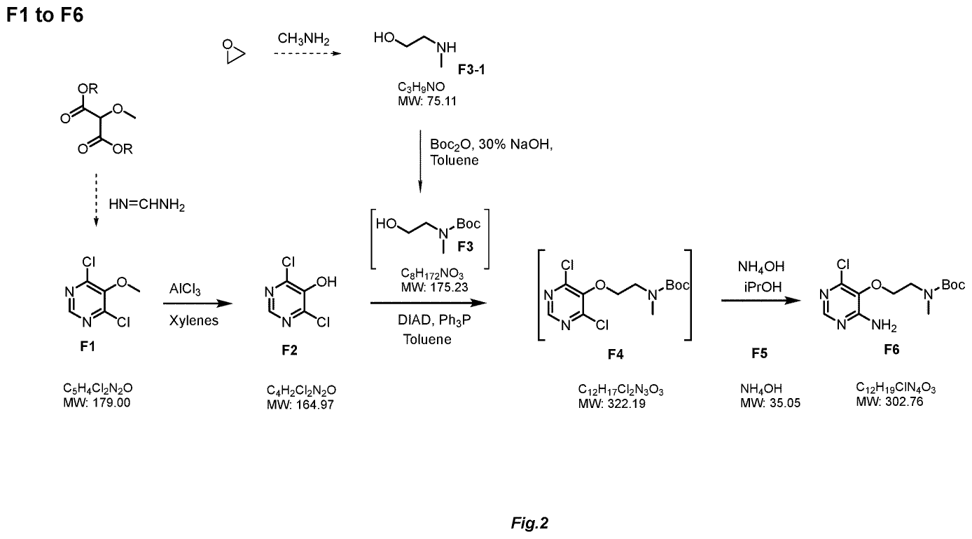

FIG. 3 demonstrates exemplary reaction conditions for the route from N6e to X6b.

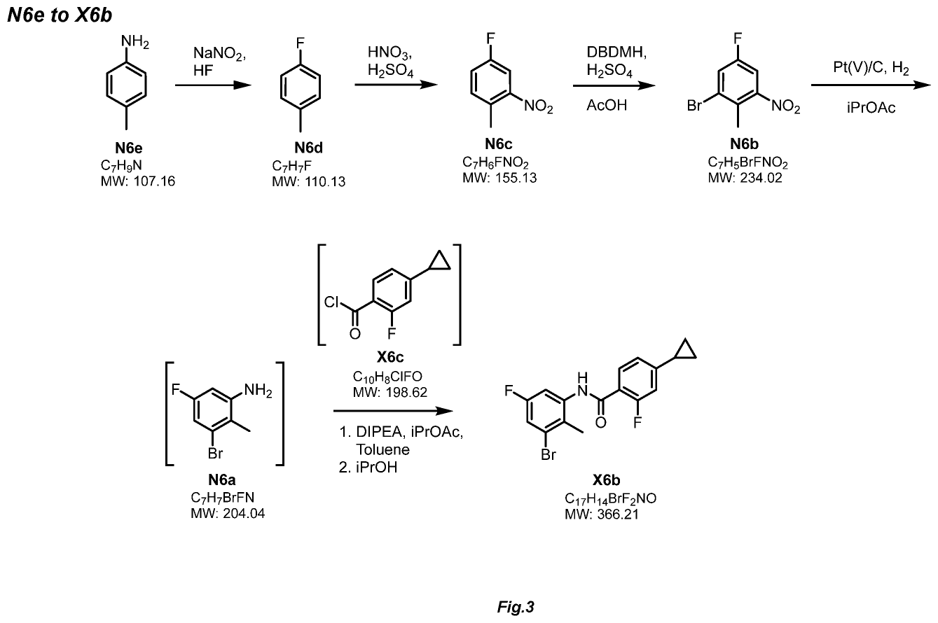

FIG. 4 demonstrates exemplary reaction conditions for the route from X6i to X6b.

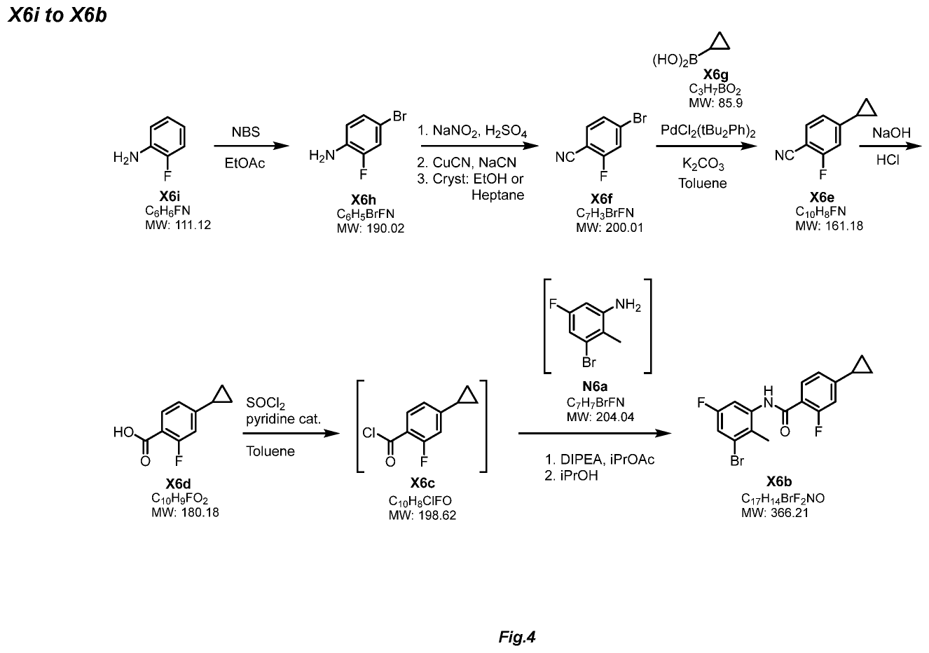

FIG. 5 demonstrates exemplary reaction conditions for the route from X6b to F11.

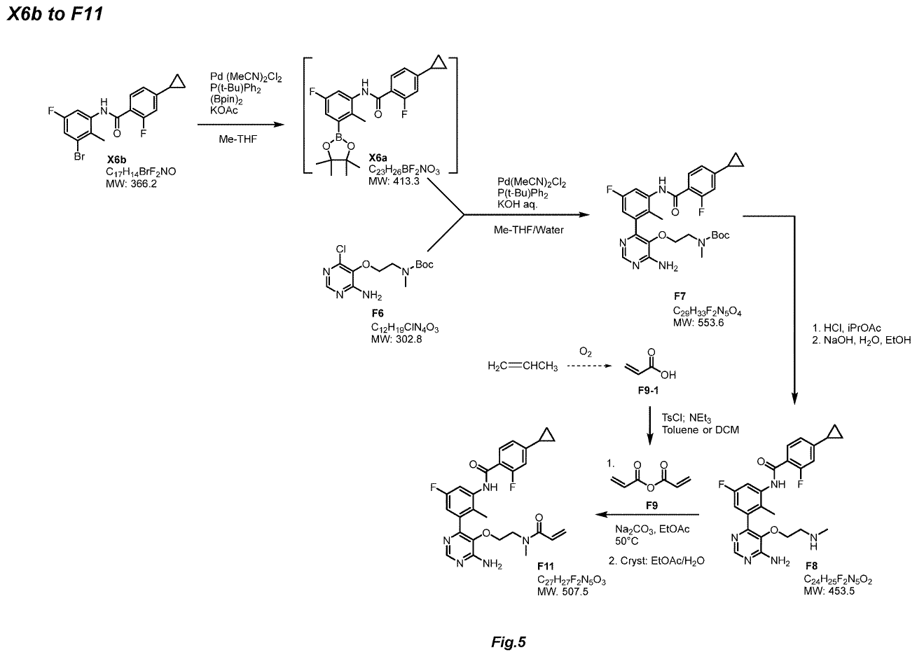

### DETAILED DESCRIPTION

The invention is useful in the preparation of N-(3-(6-Amino-5-(2-(N-methylacrylamido)ethoxy)pyrimidin-4-yl)-5-fluoro-2-methylphenyl)-4-cyclopropyl-2-fluorobenzamide, also known as remibrutinib, is a highly potent and selective oral Bruton's tyrosine kinase (BTK) inhibitor:

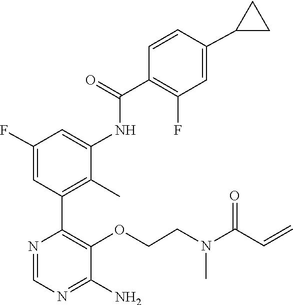 [SMILES: C=CC(=O)N(C)CCOc1c(N)ncnc1-c1cc(F)cc(NC(=O)c2ccc(C3CC3)cc2F)c1C]

In any embodiment herein, remibrutinib or any other compound described herein may be provided as a salt. As used herein, the terms “salt” or “salts” refers to an acid addition or base addition salt of a compound of the invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto. Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and trimethamine. The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Many organic solvents are suitable for the chemical reactions described herein. For example, the reactions described herein may be conducted in an aprotic organic solvent. Suitable examples include: acetonitrile; dimethylsulfoxide (DMSO); dimethylformamide (DMF); halogenated alkanes such as dichloromethane (DCM); aromatic compounds such as benzene, toluene, xylene, mesitylene, and naphthalene; alkanes such as hexane, heptane, and octane; ketones such as acetone; ether compounds such as diethyl ether, tetrahydrofuran (THF), derivatives of THF such as methyl-THF; ester compounds such as ethyl acetate and isopropylacetate; amines such as pyridine; polyethylene glycol (PEG); in particular PEG with an average molecular weight of about 100 g/mol to about 2000 g/mol such as PEG200, PEG600, PEG1000 and PEG2000, derivatives thereof such as mono- or dialkyl PEG, in particular mono- or dimethyl PEG, mono- or diethyl PEG and mono- or dipropyl PEG; and polypropylene glycol (PPG). Protic solvents may also be used in the reactions described herein. Protic solvents include: water; alcohols such as a C₁₋₁₀ aliphatic branched or linear alcohols, in particular C₁-C₆ alcohols; and carboxylic acids such as methanoic acid, ethanoic acid, propanoic acid, etc. Preferred solvents include toluene, ethanol, ethyl acetate isopropyl acetate, methyl-THF, heptane and isopropanol. Preferably, the reactions described herein are carried out avoiding non-desirable solvents such as DCM, DME, DMF, dioxane and 1,2 dichloroethane or other carcinogenic or teratogenic solvents. In certain embodiments, the amount of solvent in the reaction mixture is in the range of from 0.1% to 99% (v/v), from 0.1% to 80% (v/v), from 0.1% to 75% (v/v), from 0.1% to 50% (v/v), from 1% to 40% (v/v), from 2% to 30% (v/v), from 4% to 25% (v/v) or from 5% to 20% (v/v).

Some chemical reactions described herein can be conducted under acidic conditions, e.g. at a pH of less than 7, no more than 6, no more than 5, no more than 4, no more than 3, no more than 2, or no more than 1. Acids suitable for the chemical reactions described are known to the skilled person. Commonly used acids include inorganic acids, for example sulfuric acid, phosphoric acid, and nitric acid, boric acid; halo acids such as hydrofluoric acid, hydrochloric acid, hydrobromic acid, and hydroiodic acid; organic acids, for example carboxylic acids and derivatives thereof such as acetic acid, benzoic acid; and halogenated acetic acids such as trifluoroacetic acid, and dichloroacetic acid. Preferably, the acid is HF, HCl, or H₂SO₄. Preferably, fluorinated acids such as TFA are avoided in order to avoid generation of fluorinated waste.

Some chemical reactions described herein can be conducted under basic conditions, e.g. at a pH of greater than 7, at least 8, at least 9, at least 10, at least 11, at least 12, at least 13, or at least 14. Basic compounds suitable for the chemical reactions described herein are known to the skilled person. Commonly used bases include inorganic bases, for example hydroxides of alkali metals and alkali earth metals such as lithium hydroxide, sodium hydroxide, potassium hydroxide, magnesium hydroxide, and calcium hydroxide. Stronger bases can be made from the addition of alkali earth metals to hydrocarbons, amines and dihydrogen. Examples include butyl lithium, lithium diisopropylamide (LDA), Lithium diethylamide (LDEA), sodium amide, sodium hydride (NaH), and Lithium bis(trimethylsilyl)amide. Weaker bases include ammonia and amines, for example trialkylamines such as triethylamine and diisopropylethylamine, and anions of weak acids such as acetates (e.g. sodium acetate), potassium acetate, and carbonates (e.g. sodium carbonate, potassium carbonate).

The reactions described herein can be run for as long as needed to achieve completion of the reaction, or at least an acceptable yield of product. For example, the duration of the reaction may be less than 1 minute, less than 5 minutes, less than 10 minutes, less than 30 minutes, less than 1 hour, less than 2 hours, less than 3 hours, less than 5 hours, less than 10 hours, less than 20 hours, less than 30 hours less than 40 hours, less than 50 hours, or less than 60 hours. The reaction time may depend, inter alia, on the scale of the reaction. The skilled person can monitor the progress of the reaction in a number of different ways including by monitoring physical changes such as a change in colour, or by monitoring the reaction using analytical methods such as NMR, FT-IR, XRPD, or chromatography, for example thin layer chromatography (TLC) or liquid chromatography coupled to mass spectrometry (LC-MS).

Upon completion of the reactions described herein, the reaction mixture is optionally purified. Purification techniques are known to the skilled person and include: chromatography (e.g. HPLC, which may be reverse phase or normal phase); liquid-liquid separation, for example using multiple immiscible solvents; and/or liquid-solid separation, for example using filtration, decantation, (re)crystallisation, trituration, evaporation, freeze-drying.

The reactions described herein may be performed on any suitable scale. In one embodiment, the reaction mixture is of industrial scale. It may for example have a volume of at least 1 l, in particular at least 10 l, at least 100 l, or at least 1000 l. In another embodiment, the reaction mixture is of microscale. It may for example have a volume of 10 ml or less, in particular 1 ml or less, 100 μl or less, 10 μl or less or 1 μl or less.

The reactions described herein may be part of a series of reactions comprising a synthesis. Where multiple reactions are described, these can be conducted in a sequential fashion or in a one-pot fashion. Sequential reactions typically involve the completion of a first reaction, followed by work up and purification of that reaction, before a second reaction is conducted, continuing with further reactions until the desired product has been made. In contrast, in a one-pot fashion, a first reaction may be completed, and then a second reaction may be conducted using one or more products of the first reaction without isolation. One-pot reactions are advantageous because they avoid unnecessary purification steps, saving time and materials. In the synthesis of remibrutinib described herein, some or all reactions may be conducted in a one-pot fashion, or alternatively some or all reactions may be conducted in a sequential fashion.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements may and/or indeed does encompass further elements as well as embodiments wherein the subject-matter which “comprises” specifically listed elements does not comprise further elements.

Numeric ranges described herein are inclusive of the numbers defining the range. The headings provided herein are not limitations of the various aspects or embodiments of this invention which can be read by reference to the specification as a whole. According to one embodiment, subject matter described herein as comprising certain steps in the case of methods or as comprising certain ingredients in the case of compositions refers to subject matter consisting of the respective steps or ingredients. It is preferred to select and combine specific aspects and embodiments described herein and the specific subject-matter arising from a respective combination of specific embodiments also belongs to the present disclosure.

The present invention provides a new synthetic route to remibrutinib which avoids the formation of genotoxic intermediate INT 3. The key to avoiding INT 3 is to prepare aryl halide X6b, which does not contain a boronic ester and therefore can be synthesised via N6a instead of INT 3, as set out below. Moreover, the claimed process minimizes purification steps, improving overall yield and providing a more efficient process. The process can also be conducted efficiently in green solvents.

### Preparation of F7

The invention provides a synthesis method comprising converting compound X6b and compound F6 into compound F7:

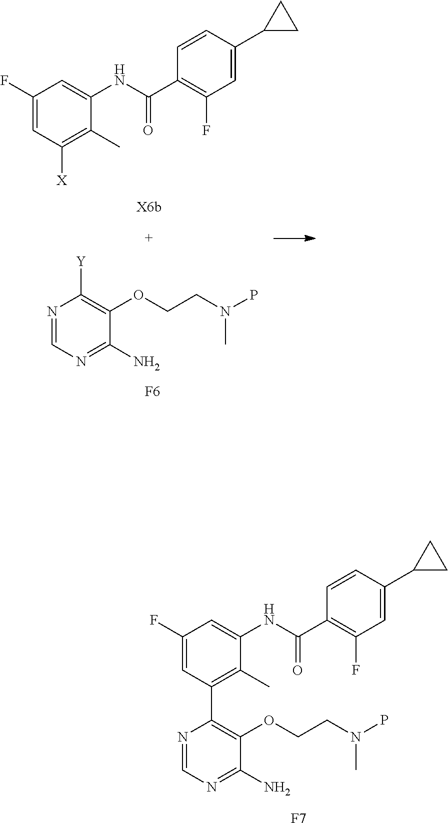

wherein X and Y are each independently Cl, Br, or I, and wherein P is an amine protecting group.

In some embodiments, X is Cl or Br. In some embodiments, Y is Cl or Br. In some embodiments, X and Y are each Cl or Br. In some embodiments, X is Br. In some embodiments, Y is Cl. In some embodiments, X is Br and Y is Cl. These embodiments apply to any and all instances of X and Y described herein, including X and Y groups present on synthetic precursors to X6b and F6, respectively.

The protecting group P can be any suitable amine protecting group that is stable during any of the chemical transformations described herein (except for deprotection steps). Amine protecting groups may be removed by particular conditions, for example acid, base, hydrogenation, light, heat, etc. Examples of suitable amine protecting groups include carbamate protecting groups, such as 9-fluorenylmethyl carbamate (Fmoc), t-butyl carbamate (Boc), or benzyl carbamate (Cbz); acetamide protecting groups such as acetamide, trifluoroacetamide, or benzylamide; and sulfonamide protecting groups such as p-toluenesulfonamide.

X6b and F6 can be converted to F7 according to coupling conditions suitable for forming a carbon-carbon bond. For example, the coupling of X6b and F6 can be achieved using an organometallic cross-coupling reaction whereby the two fragments are joined together with the aid of a metal catalyst. Cross-coupling conditions that could be employed in the coupling of X6b and F6 include: Kumada coupling; Negishi coupling; Stille coupling; Suzuki-Myaura coupling, and Hiyama coupling. In a typical cross-coupling reaction, a compound of the type R-M (R=first organic fragment, M=metal or main group compound) reacts with an organic halide of the type R′—X (R′=second organic fragment, X=halide) with formation of a new carbon-carbon bond in the product R—R′.

Thus, in some embodiments, the preparation of F7 comprises the conversion of F6 into pre-cursor F6′ by replacement of Y with “M”, a metal-containing moiety, or a main group element containing moiety, for example wherein M contains Zn (Negishi) B (Suzuki-Myaura), Mg (Kumada), Sn (Stille), or Si (Hiyama):

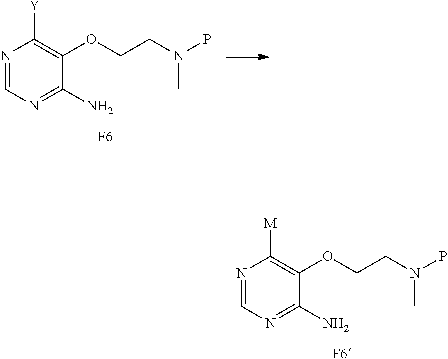

wherein P is an amine protecting group, e.g. Boc.

F6′ can be reacted with X6b under cross-coupling conditions to give F7. In some embodiments, the conversion of F6 to F6′ and the cross-coupling of F6′ with X6b is conducted in a one-pot reaction. In some embodiments, the conversion of F6 to F6′ and the cross-coupling of F6′ with X6b are conducted in sequential reactions.

Alternatively, the preparation of F7 comprises the conversion of X6b into a precursor compound X6b′ by replacement of X with “M”, a metal-containing moiety, or a main group element-containing moiety, for example wherein M contains Zn (Negishi) B (Suzuki-Miyaura), Mg (Kumada), Sn (Stille), or Si (Hiyama):

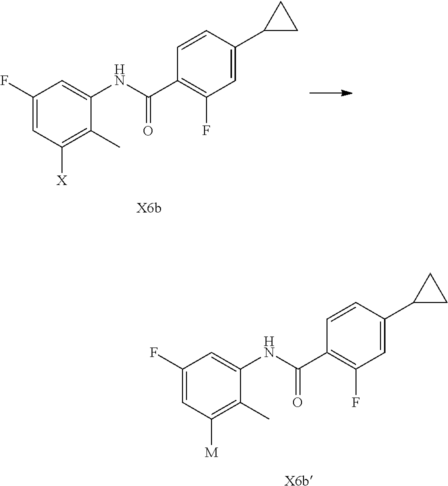

The precursor compound X6b′ can be reacted with F6 under cross-coupling conditions to give F7. In some embodiments, the conversion of X6b to X6b′ and the cross-coupling of X6b′ with F6 is conducted in a one-pot reaction. In some embodiments, the conversion of X6b to X6b′ and the cross-coupling of X6b′ with F6 are conducted in sequential reactions.

### Preparation of X6a—Borylation Reaction

The invention provides a synthesis method comprising the borylation of X6b to give X6a:

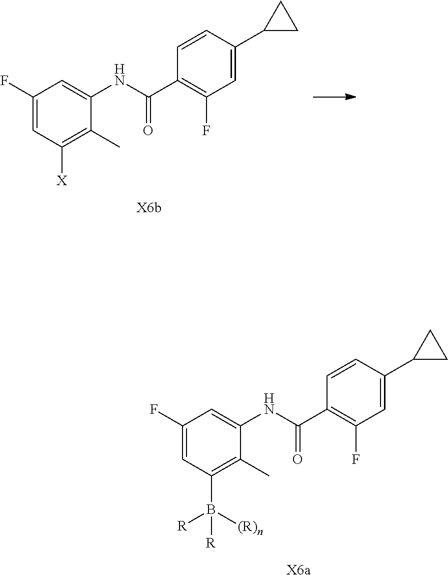

wherein X is F, Cl, Br, or I; n is 0 or 1 and R is F, Cl, Br, or I, OH, OC₁-C₆alkyl, N(C₁-C₆ alkyl)₂, aryl, or wherein two or three R groups other than F, Cl, Br, I, or OH can be taken together to form a cyclic boronate ester, for example pinacol boronate, or N-methyliminodiacetic acid (MIDA) boronate.

The borylation of X6b can be achieved using one or more catalysts, one or more ligands, one or more borylating agents, one or more bases, and/or one or more additives. In some embodiments, the borylation includes one or more catalysts, one or more ligands, one or more borylating agents, and one or more bases. In some embodiments, the borylation additionally includes one or more additives.

Borylating agents are boron-containing compounds that are capable of converting organohalide compounds into boronic acids or boronic esters, usually under metal-catalysed cross-coupling conditions. In some embodiments, the borylating agent is selected from the group consisting of diboron compounds, boronic acids, boranes, boron trihalides, and borates. In some embodiments, the borylating agent is selected from the group consisting of bis(pinacolato)diboron, B₂(NMe₂)₄, B₂F₄, B₂Cl₄, B₂Br₄, B₂I₄, bis-boronic acid, pinacolborane, HB(NMe₂)₂, B(OH)₃, BF₃, BCl₃, BBr₃, BI₃, mono-, di-, or tri-C₁-C₆alkylborate, mono-, di-, or tri-methylborate, mono-, di-, or tri-ethylborate, and mono-, di-, or tri-propylborate, preferably bis(pinacolato)diboron or bis-boronic acid. The use of bis-boronic acid may be attractive as it can allow for lower catalyst loadings, milder reaction conditions, and avoids the formation of pinacol-related impurities, as compared to pinacolborane or bis(pinacolato)diboron. It also allows for the use of green solvent such as alcoholic solvent and milder reaction condition (e.g. lower temperature).

The metal catalyst used in the borylation reaction may contain palladium, nickel, or copper, or a combination thereof, preferably palladium.

In some embodiments, the metal catalyst is provided as a pre-catalyst complex, for example a Buchwald G1, G2, G3, or G4 pre-catalyst complexed to a phosphine ligand. Buchwald pre-catalysts are used to generate active Pd(0) in situ via rapid deprotonation and reductive elimination. Pre-catalysts are useful as they allow low catalytic loadings and are stable to air, moisture and heat with good solubility. These pre-catalysts have been optimised to further enhance function and solubility from Generations 1 to 4 (G1 to G4). The pre-catalysts comprise of a palladacycle (shown below) with a phenyl or 1,1-biphenyl backbone, wherein L represents a bound phosphine ligand eg. XPhos, SPhos, etc. (see below) and wherein the bound amine substituents and the leaving group (Cl, OMs) vary based on the Generation.

Examples of Buchwald pre-catalysts complexed with palladium and with an exemplary XPhos ligand are shown below:

| | Pre-catalyst | | Complex with XPhos | |
|---|---|---|---|---|
| G# | Formula | Structural name | Formula | Structural name |
| G1 | 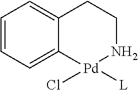 | chloropalladium(1+); (L); 2- phenylethanamine | 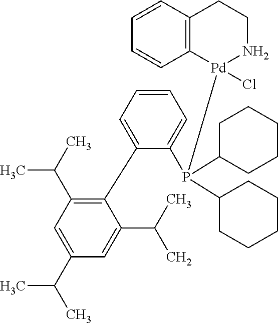 | chloropalladium(1+); dicyclohexyl-[2-[2,4,6- tri(propan-2- yl)phenyl]phenyl] phosphane; 2- phenylethanamine |
| G2 | 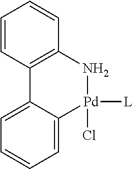 | chloropalladium(1+); (L); 2-phenylaniline | 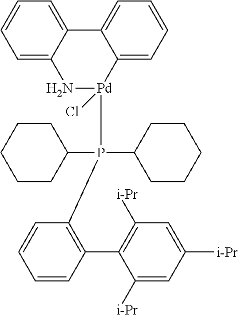 | chloropalladium(1+); dicyclohexyl-[2-[2,4,6- tri(propan-2- yl)phenyl]phenyl] phosphane; 2- phenylaniline |
| G3 | 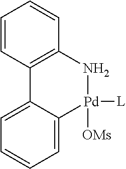 | (L); methanesulfonate; palladium(2+); 2- phenylaniline | 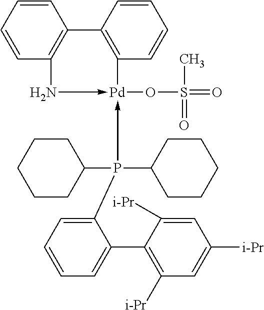 | dicyclohexyl-[2- [2,4,6-tri(propan-2- yl)phenyl]phenyl] phosphane; methane- sulfonate; palladium(2+); 2-phenylaniline |
| G4 | 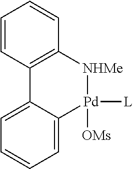 | (L); methanesulfonate; N-methyl-2- phenylaniline; palladium(2+) | 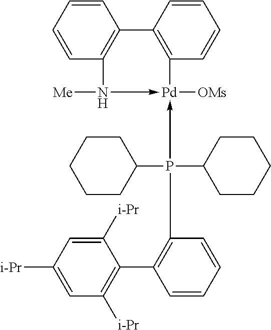 | dicyclohexyl-[2- [2,4,6-tri(propan-2- yl)phenyl]phenyl] phosphane; methane- sulfonate; N-methyl-2- phenylaniline; palladium(2+) |

Any other phosphine ligand described herein may be used as L instead of XPhos in the table above.

Other precatalysts for borylation may include Pd(TFA)₂, PdBr₂ or Pd(MeCN)₂Cl₂. This precatalysts ca be used in the presence of ligands such as Ph₂P(t-Bu); Cy₃P—HBF₄; RuPHOS; S—PHOS, Cy-BIPHEP; SPHOS—SO₃Na.

In some embodiments, the borylation of X6b involves a further ligand in addition to the ligand L forming part of the pre-catalyst complex. In other embodiments, no further ligand is required. In some embodiments, the borylation of X6b uses a catalyst and a ligand without a pre-catalyst (Pd(O) catalyst; e.g. Pd(PPh₃)₄).

A wide range of ligands can be used in borylation reactions, and the ligand can affect the reactivity of the reagents. For example, ligands can increase the electron density at the metal center of the metal complex, which can improve the oxidative addition step. In addition, a bulky ligand helps in the reductive elimination step. In some embodiments, the ligand used in the borylation of X6b is selected from the group consisting of organophosphines, N-heterocyclic carbenes, diazabutadiene, dibenzylideneacetone, and combinations thereof.

In a preferred embodiment, the ligand is an organophosphine ligand, for example an organophosphine selected from the group consisting of XPhos, APhos, CPhos, RuPhos, SPhos, cataCXium, DavePhos, JohnPhos, MePhos, XantPhos, Cy₃P—HBF₄, Cy-BIPHEP, SPhos-SO₃Na, PPh₃, tBuPPh₂ and combinations thereof, preferably XPhos, APhos, CPhos, RuPhos, SPhos, cataCXium, more preferably XPhos, cataCXium and tBuPPh₂, most preferably tBuPPh₂. Phosphine ligands are depicted in the table below:

| Phosphine | Formula | IUPAC Name |
|---|---|---|
| XPhos | 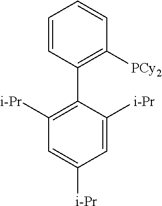 | dicyclohexyl-[2-[2,4,6-tri(propan-2- yl)phenyl]phenyl]phosphane |
| RuPhos | 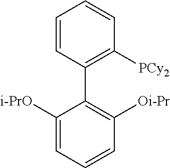 | dicyclohexyl-[2-[2,6-di(propan-2- yloxy)phenyl]phenyl]phosphane |
| SPhos | 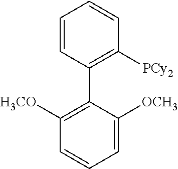 | dicyclohexyl-[2-(2,6- dimethoxyphenyl)phenyl]phosphane |
| DavePhos | 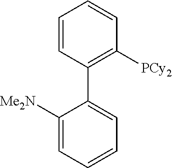 | 2-(2-dicyclohexylphosphanylphenyl)-N,N- dimethylaniline |
| JohnPhos | 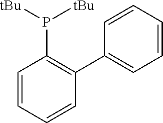 [SMILES: CC(C)(C)P(c1ccccc1-c1ccccc1)C(C)(C)C] | ditert-butyl-(2-phenylphenyl)phosphane |
| MePhos | 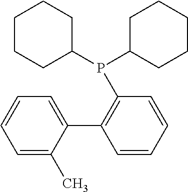 [SMILES: Cc1ccccc1-c1ccccc1P(C1CCCCC1)C1CCCCC1] | dicyclohexyl-[2-(2- methylphenyl)phenyl]phosphane |
| CataCXium | 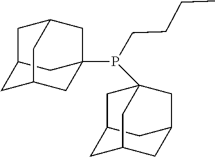 [SMILES: CCCCP(C12CC3CC(CC(C3)C1)C2)C12CC3CC(CC(C3)C1)C2] | bis(1-adamantyl)-butylphosphane |
| APhos | 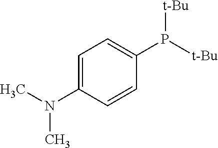 [SMILES: CN(C)c1ccc(P(C(C)(C)C)C(C)(C)C)cc1] | 4-ditert-butylphosphanyl-N,N- dimethylaniline |
| XantPhos | 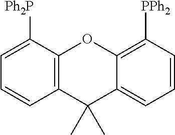 | (5-diphenylphosphanyl-9,9- dimethylxanthen-4-yl)-diphenylphosphane |
| PPh₃ | 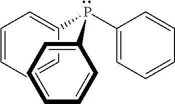 | triphenylphosphine |
| t-BuPPh2 | 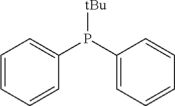 [SMILES: CC(C)(C)P(c1ccccc1)c1ccccc1] | Tertbutyldiphenylphosphine |
| PCy3•HBF4 | 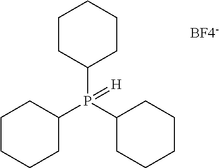 | Tricyclohexylphosphine tetrafluoroborate |
| Cy-BIPHEP | 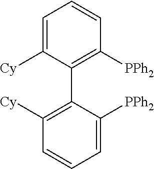 | (6,6′-Dicyclohexylbiphenyl-2,2′-diyl)-bis- (diphenylphosphine) |

The borylation of X6b can involve a base. In some embodiments, the base is an organic or inorganic salt such as NaOH, Ca(OH)₂, Na₂CO₃, K₂CO₃, K₃PO₄, Cs₂CO₃, KOAc, KOPh, or NaOAc, a tertiary amine, such as diisopropylethylamine (DIPEA), triethylamine, or a combination thereof. Preferably, the base is DIPEA, KOAc or KOH, most preferably KOAc.

The borylation of X6b can involve an additive, for example an alcohol such as ethylene glycol. In some embodiments, the borylation of X6b does not involve an additive.

The borylation of X6b can be conducted in any suitable solvent. Examples of suitable organic solvents include polar solvents, non-polar solvents, protic solvents, aprotic solvents, polar protic solvents, and polar aprotic solvents. In one embodiment, borylation can be conducted in alcoholic solvents, including t-amyl alcohol, hexanol, pentanol, butanol (tert-butanol, isobutanol, and n-butanol), propanol (isopropanol and n-propanol), ethanol, and/or methanol. Preferably, the borylation is conducted in methanol, toluene, and/or MeTHF, most preferably in MeTHF. Other solvents can also be used, for example halogenated alkane solvents such as dichloromethane. Ether-based solvents such as dioxane, MeTHF, THF, and dialkylethers such as diethylether, can also be used. Borylation can also be conducted in an aqueous environment, including a micellar environment. In some embodiments, a mixture of solvents is used.

The borylation of X6b can be achieved using one or more catalysts, one or more ligands, one or more borylating agents, one or more bases, and/or optionally one or more additives. The skilled person can determine appropriate amounts of these reagents. Nonetheless, in some embodiments of the borylation reaction:
i) The catalyst or pre-catalyst is present in an amount of 0.01 mol % to 3 mol %, 0.05 mol % to 2 mol %, 0.1 mol % to 2 mol %, 0.1 to 1 mol %, preferably 0.25 mol % and more preferably 0.5 mol % relative to the number of moles of X6b;
ii) The ligand is in an amount of 0.02 mol % to 6 mol %, 0.1 mol % to 2 mol %, 0.2 mol % to 1 mol %, 0.5 mol % or 1 mol % relative to the number of moles of X6b;
iii) The number of moles of ligand is twice or 3 times the number of moles of catalyst or pre-catalyst; preferably twice;
iv) The borylating agent is in an amount of 1 to 3 molar equivalents compared to X6b, preferably in an amount of 1 to 2 molar equivalent, more preferably 1.05 or 1.5 molar equivalents compared to X6b;
v) The base is in an amount of 2 to 5 molar equivalents, preferably 2 to 3 molar equivalent, most preferably 2.5 or 3 molar equivalents relative to the number of moles of X6b; and/or
vi) The additive is optional and when present is in an amount of 2 to 5 molar equivalents compared to X6b; preferably the additive is absent.

The borylation reaction may be characterised by any one of i) to vi) above. The borylation reaction may be characterised by any two of i) to vi) above. The borylation reaction may be characterised by any three of i) to vi) above. The borylation reaction may be characterised by any four of i) to vi) above. The borylation reaction may be characterised by any five of i) to vi) above. The borylation reaction may be characterised by all of i) to vi) above.

The borylation reaction may be characterised by i) and ii) above. The borylation reaction may be characterised by i) and iii) above. The borylation reaction may be characterised by i) and iv) above. The borylation reaction may be characterised by i) and v) above. The borylation reaction may be characterised by i) and vi) above. The borylation reaction may be characterised by ii) and iii) above. The borylation reaction may be characterised by ii) and iv) above. The borylation reaction may be characterised by ii) and v) above. The borylation reaction may be characterised by ii) and vi) above. The borylation reaction may be characterised by iii) and iv) above. The borylation reaction may be characterised by iii) and v) above. The borylation reaction may be characterised by iii) and vi) above. The borylation reaction may be characterised by iv) and v) above. The borylation reaction may be characterised by iv) and vi) above. The borylation reaction may be characterised by v) and vi) above.

In one example, a borylation reaction having excellent yield and minimal by-products may be:

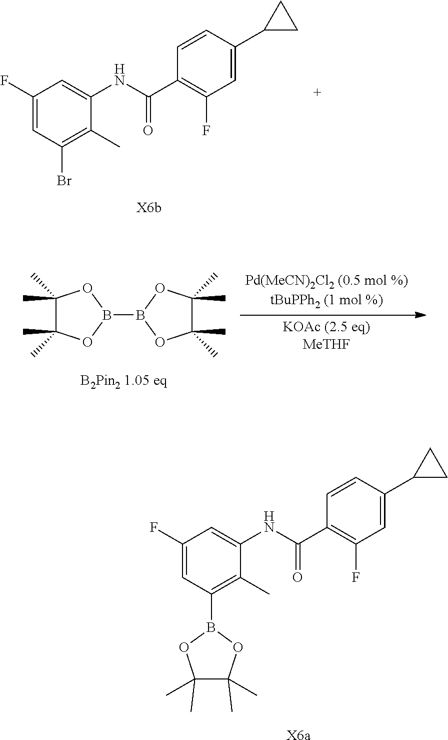

In one embodiment, a borylation reaction having excellent yield and minimal-by product formation is characterized by at least one of the following:
i) the catalyst is Pd(MeCN)₂Cl₂ in an amount of 0.1 mol % to 2 mol % relative to the number of moles of X6b, or 0.1 mol % to 1.5 mol %, preferably 0.25 mol % or more preferably 0.5 mol % relative to the number of moles of X6b;
ii) the ligand is tBuPPh₂ in an amount of 0.2 mol % to 4% relative to the number of moles of X6b, 0.2 mol % to 3 mol %, preferably 0.5 mol % or more preferably 1 mol % relative to the number of moles of X6b;
iii) the catalyst is Pd(MeCN)₂Cl₂, the ligand is tBuPPh₂ and the number of moles of tBuPPh₂ is twice or three times the number of moles of Pd(MeCN)₂Cl₂, preferably twice the number of moles of Pd(MeCN)₂Cl₂;
iv) the borylating agent is bis(pinacolato)diboron in an amount of 1 to 2 molar equivalents relative to X6b, preferably about 1.05 molar equivalents relative to X6b;
v) the base is KOAc in an amount of 2 to 5 molar equivalents relative to X6b, preferably 2.5 equivalents relative to X6b; and
vi) no additive is present; and/or
vii) the temperature of the reaction is 30° C. to 120° C., e.g. 40° C. to 50° C., preferably 60° C. or 70° C.

The borylation reaction may be characterised by any one of i) to vii) above. The borylation reaction may be characterised by any two of i) to vi) above. The borylation reaction may be characterised by any three of i) to vii) above. The borylation reaction may be characterised by any four of i) to vii) above. The borylation reaction may be characterised by any five of i) to vii) above. The borylation reaction may be characterised by any six of i) to vii) above. The borylation reaction may be characterised by all of i) to vii) above.

The borylation reaction may be characterised by i) and ii) above. The borylation reaction may be characterised by i) and iii) above. The borylation reaction may be characterised by i) and iv) above. The borylation reaction may be characterised by i) and v) above. The borylation reaction may be characterised by i) and vi) above. The borylation reaction may be characterised by i) and vii) above. The borylation reaction may be characterised by ii) and iii) above. The borylation reaction may be characterised by ii) and iv) above. The borylation reaction may be characterised by ii) and v) above. The borylation reaction may be characterised by ii) and vi) above. The borylation reaction may be characterised by ii) and vii) above. The borylation reaction may be characterised by iii) and iv) above. The borylation reaction may be characterised by iii) and v) above. The borylation reaction may be characterised by iii) and vi) above. The borylation reaction may be characterised by iii) and vii) above. The borylation reaction may be characterised by iv) and v) above. The borylation reaction may be characterised by iv) and vi) above. The borylation reaction may be characterised by iv) and vii) above. The borylation reaction may be characterised by v) and vi) above. The borylation reaction may be characterised by v) and vii) above. The borylation reaction may be characterised by vi) and vi) above.

In one embodiment, a borylation reaction having good yield and minimal by-product formation is characterised by at least one of the following:
i) the catalyst is a pre-catalyst which is Pd-XPhos-2G in an amount of is 0.05 mol % to 0.5 mol % relative to the number of moles of X6b, preferably 0.25 mol % relative to the number of moles of X6b;
ii) the ligand is XPhos in an amount of 0.1 mol % to 1 mol % relative to the number of moles of X6b; preferably 0.5 mol % relative to the number of moles of X6b;
iii) the catalyst is Pd-XPhos-2G, the ligand is XPhos, and the number of moles of XPhos is twice the number of moles of Pd-XPhos-2G;
iv) the borylating agent is bis-boronic acid in an amount of 1 to 3 molar equivalents compared to X6b, preferably 1.5 molar equivalents compared to X6b;
v) the base is potassium acetate in an amount of 2 to 5 molar equivalents, preferably 3 molar equivalents relative to X6b;
vi) the additive is ethylene glycol in an amount of 2 to 5 molar equivalents compared to X6b, preferably 3 molar equivalents relative to X6b; and
vii) the temperature of the reaction is 30° C. to 70° C., preferably 40° C. to 50° C., more preferably 50° C.

The borylation reaction may be characterised by any one of i) to vii) above. The borylation reaction may be characterised by any two of i) to vi) above. The borylation reaction may be characterised by any three of i) to vii) above. The borylation reaction may be characterised by any four of i) to vii) above. The borylation reaction may be characterised by any five of i) to vii) above. The borylation reaction may be characterised by any six of i) to vii) above. The borylation reaction may be characterised by all of i) to vii) above.

The borylation reaction may be characterised by i) and ii) above. The borylation reaction may be characterised by i) and iii) above. The borylation reaction may be characterised by i) and iv) above. The borylation reaction may be characterised by i) and v) above. The borylation reaction may be characterised by i) and vi) above. The borylation reaction may be characterised by i) and vii) above. The borylation reaction may be characterised by ii) and iii) above. The borylation reaction may be characterised by ii) and iv) above. The borylation reaction may be characterised by ii) and v) above. The borylation reaction may be characterised by ii) and vi) above. The borylation reaction may be characterised by ii) and vii) above. The borylation reaction may be characterised by iii) and iv) above. The borylation reaction may be characterised by iii) and v) above. The borylation reaction may be characterised by iii) and vi) above. The borylation reaction may be characterised by iii) and vii) above. The borylation reaction may be characterised by iv) and v) above. The borylation reaction may be characterised by iv) and vi) above. The borylation reaction may be characterised by iv) and vii) above. The borylation reaction may be characterised by v) and vi) above. The borylation reaction may be characterised by v) and vii) above. The borylation reaction may be characterised by vi) and vii) above.

In another example, the borylation reaction may be:

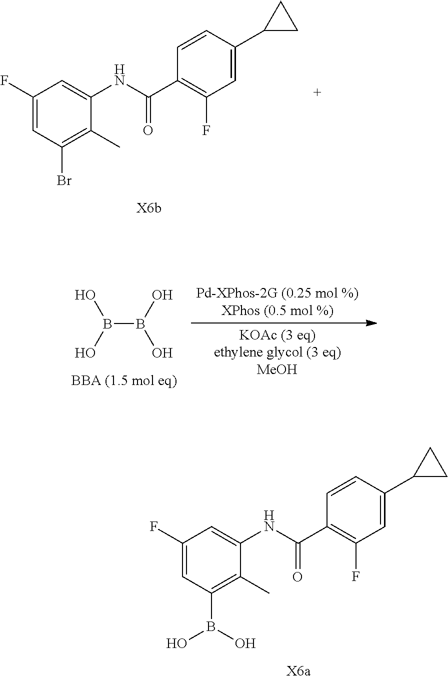 [SMILES: Cc1c(Br)cc(F)cc1NC(=O)c1ccc(C2CC2)cc1F ; "X6b" ; OB(O)B(O)O ; Cc1c(NC(=O)c2ccc(C3CC3)cc2F)cc(F)cc1B(O)O ; "X6a"]

In one embodiment, a borylation reaction having good yield and minimal by-product formation is characterised by at least one of the following:
i) the catalyst is Pd-cataCXium-3G in an amount of 0.001 mol % to 0.5 mol % relative to the number of moles of X6b, preferably 0.05 mol % relative to the number of moles of X6b;
ii) the ligand is cataCXium in an amount of 0.02 mol % to 1% relative to the number of moles of X6b, preferably 0.1 mol % relative to the number of moles of X6b;
iii) the catalyst is Pd-cataCXium-3G, the ligand is cataCXium and the number of moles of cataCXium is twice the number of moles of Pd-cataCXium-3-3G;
iv) the borylating agent is bis-boronic acid in an amount of 1 to 3 molar equivalents relative to X6b, preferably 1.5 molar equivalents relative to X6b;
v) the base is N,N-diisopropylethylamine in an amount of 2 to 5 molar equivalents relative to X6b, preferably equivalents relative to X6b; and
vi) no additive is present; and/or
vii) the temperature of the reaction is 30° C. to 70° C., preferably 40° C. to 50° C., more preferably 50° C.

The borylation reaction may be characterised by any one of i) to vii) above. The borylation reaction may be characterised by any two of i) to vi) above. The borylation reaction may be characterised by any three of i) to vii) above. The borylation reaction may be characterised by any four of i) to vii) above. The borylation reaction may be characterised by any five of i) to vii) above. The borylation reaction may be characterised by any six of i) to vii) above. The borylation reaction may be characterised by all of i) to vii) above.

The borylation reaction may be characterised by i) and ii) above. The borylation reaction may be characterised by i) and iii) above. The borylation reaction may be characterised by i) and iv) above. The borylation reaction may be characterised by i) and v) above. The borylation reaction may be characterised by i) and vi) above. The borylation reaction may be characterised by i) and vii) above. The borylation reaction may be characterised by ii) and iii) above. The borylation reaction may be characterised by ii) and iv) above. The borylation reaction may be characterised by ii) and v) above. The borylation reaction may be characterised by ii) and vi) above. The borylation reaction may be characterised by ii) and vii) above. The borylation reaction may be characterised by iii) and iv) above. The borylation reaction may be characterised by iii) and v) above. The borylation reaction may be characterised by iii) and vi) above. The borylation reaction may be characterised by iii) and vii) above. The borylation reaction may be characterised by iv) and v) above. The borylation reaction may be characterised by iv) and vi) above. The borylation reaction may be characterised by iv) and vii) above. The borylation reaction may be characterised by v) and vi) above. The borylation reaction may be characterised by v) and vii) above. The borylation reaction may be characterised by vi) and vii) above.

For example, the reaction may be:

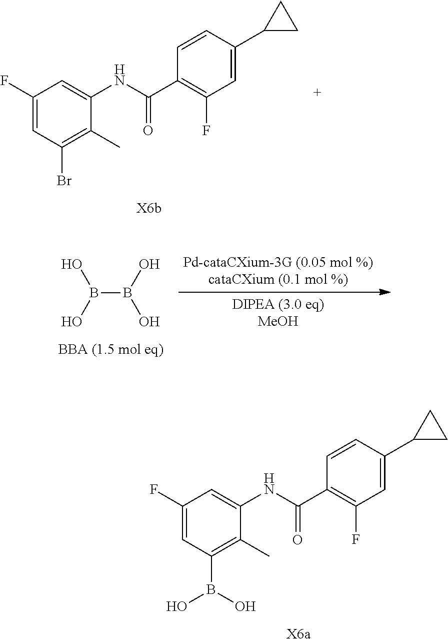 [SMILES: Cc1c(Br)cc(F)cc1NC(=O)c1ccc(C2CC2)cc1F ; "X6b" ; OB(O)B(O)O ; Cc1c(NC(=O)c2ccc(C3CC3)cc2F)cc(F)cc1B(O)O ; "X6a"]

### Coupling of X6a and F6

In some embodiments of the invention, the borylation of X6b to give X6a is used in a method of synthesising compound F7. In such embodiments, X6b is converted to X6a, and then X6a is reacted with F6 under cross-coupling conditions to generate F7. In a preferred embodiment, the conversion of X6b to X6a and the cross-coupling of X6a with F6 is conducted in a one-pot reaction. In some embodiments, the conversion of X6b to X6a and the cross-coupling of X6a with F6 are conducted in sequential reactions.

According to the invention, borylated compound X6a can be reacted with aryl halide in a cross-coupling reaction. In one embodiment, the coupling reaction is conducted using one or more catalysts, one or more ligands, one or more bases, and/or one or more additives. In one embodiment, the coupling reaction is conducted using one or more catalysts, one or more ligands, and one or more bases. In some embodiments, the coupling additionally includes one or more additives.

The metal catalyst used in the cross-coupling reaction may contain palladium, nickel, or copper, or a combination thereof, preferably palladium.

A wide range of ligands can be used in the cross-coupling of X6a and F6, and the ligand can affect the reactivity of the coupling reagents. For example, ligands can increase the electron density at the metal center of the metal complex, which can improve the oxidative addition step. In addition, a bulky ligand helps in the reductive elimination step. In some embodiments, the ligand used in the coupling of X6a and F6 is selected from the group consisting of organophosphines, N-heterocyclic carbenes, diazabutadiene, dibenzylideneacetone, and combinations thereof. In a preferred embodiment, the ligand is an organophosphine ligand, for example an organophosphine selected from the group consisting of XPhos, APhos, CPhos, RuPhos, SPhos, cataCXium, DavePhos, JohnPhos, MePhos, XantPhos, PPh₃, tBuPPh₂ and combinations thereof, preferably XPhos, APhos, CPhos, RuPhos, SPhos, cataCXium, more preferably XPhos, cataCXium and tBuPPh₂, most preferably tBuPPh₂.

In the coupling of X6a and F6, the metal catalyst and ligand may be provided as a pre-catalyst complex, for example a Buchwald G1, G2, G3, or G4 pre-catalyst, preferably G2, complexed to a phosphine ligand, for example an organophosphine selected from the group consisting of XPhos, APhos, CPhos, RuPhos, SPhos, cataCXium, DavePhos, JohnPhos, MePhos, XantPhos, Cy₃P—HBF₄, Cy-BIPHEP, SPHOS—SO₃Na, PPh₃, tBuPPh₂ and combinations thereof, preferably XPhos, APhos, CPhos, RuPhos, SPhos, cataCXium, more preferably XPhos, cataCXium and tBuPPh₂, most preferably tBuPPh₂.

In some embodiments, a pre-catalyst containing a phosphine ligand is used and no additional phosphine ligand is used. Alternatively, a pre-catalyst containing a phosphine ligand is used and additional phosphine ligand is also used. Examples of pre-catalysts for cross-coupling reaction may include Pd(TFA)₂, PdBr₂ or Pd(MeCN)₂Cl₂. These pre-catalysts can be used in the presence of ligands such as Ph₂P(t-Bu); Cy₃P—HBF₄; RuPHOS; S—PHOS, Cy-BIPHEP; SPHOS—SO₃Na.

The coupling of X6a and F6 can involve a base. In some embodiments, the base is an organic or inorganic salt such as KOH, NaOH, Ca(OH)₂, Na₂CO₃, K₂CO₃, K₃PO₄, Cs₂CO₃, KOAc, KOPh, or NaOAc, a tertiary amine, such as diisopropylethylamine (DIPEA), triethylamine, or a combination thereof. Preferably, the base is triethylamine or KOH, most preferably KOH.

The coupling of X6a and F6 can optionally involve an additive, for example an alcohol such as ethylene glycol, for example when PdXPhos-2G/XPhos complex is used.

The coupling of X6a and F6 can be conducted in any suitable solvent. Examples of suitable organic solvents include polar solvents, non-polar solvents, protic solvents, aprotic solvents, polar protic solvents, and polar aprotic solvents. In a preferred embodiment, cross-coupling reaction can be conducted in alcoholic solvents, including t-amyl alcohol, hexanol, pentanol, butanol (tert-butanol, isobutanol, and n-butanol), propanol (isopropanol and n-propanol), ethanol, and/or methanol. Other solvents can also be used, for example halogenated alkane solvents such as dichloromethane. Ether-based solvents such as dioxane, MeTHF, THF, and dialkylethers such as diethylether, can also be used. The coupling can also be conducted in an aqueous environment, including a micellar environment. In some embodiments, a mixture of solvents is used, e.g. MeTHF and water. Where methanol is used, the reaction mixture may be precipitated out of, simplifying purification.

The coupling of X6a and F6 can be achieved using one or more catalysts, one or more ligands, one or more borylating agents, one or more bases, and/or one or more additives. The skilled person can use their common general knowledge to determine appropriate amounts of these reagents.

In one embodiment, a coupling reaction having excellent yield and minimal by-product formation is characterized by at least one of the following
i) The catalyst or pre-catalyst is present in an amount of 0.1 mol % to 5 mol %, 0.25 mol % to 3 mol %, 0.5 mol % to 1.5 mol %, preferably 0.5 mol % or more preferably 1 mol % relative to the number of moles of F6 or X6a;
ii) The number of moles of ligand, if present, is twice or 3 times the number of moles of catalyst or pre-catalyst, preferably twice;
iii) The molar ratio of F6:X6a is from 2:1 to 1:2, or from 1.5:1 to 1:1.5, 1.2:1 to 1:1.2 or 1:1;
iv) the additive is optional and when present is in an amount of 2 to 5 molar equivalents relative to F6 or X6a; and/or
v) The base is in an amount of 2 to 5 molar equivalents, preferably in an amount of 2 to 3 molar equivalents, most preferably in an amount of 3 molar equivalent relative to the number of moles of F6 or X6a;

The coupling reaction may be characterised by any one of i) to v) above. The coupling reaction may be characterised by any two of i) to v) above. The coupling reaction may be characterised by any three of i) to v) above. The coupling reaction may be characterised by any four of i) to v) above. The coupling reaction may be characterised by all of i) to v) above.

The coupling reaction may be characterised by i) and ii) above. The coupling reaction may be characterised by i) and iii) above. The coupling reaction may be characterised by
i) and iv) above. The coupling reaction may be characterised by i) and v) above. The coupling reaction may be characterised by ii) and iii) above. The coupling reaction may be characterised by ii) and iv) above. The coupling reaction may be characterised by ii) and v) above. The coupling reaction may be characterised by iii) and iv) above. The coupling reaction may be characterised by iii) and v) above. The coupling reaction may be characterised by iv) and v) above.

In one embodiment, a coupling reaction having good yield and minimal by-product formation is characterised by at least one of the following:
i) the catalyst and ligand are provided as a pre-catalyst-ligand complex which is Pd and X-Phos-2G in an amount of is 0.5 mol % to 2 mol % relative to the number of moles of F6 or X6a;
ii) the base is triethylamine in an amount of 2 to 5 molar equivalents, preferably 3 molar equivalents relative to F6 or X6a;
iii) the additive is ethylene glycol in an amount of 2 to 5 molar equivalents, preferably 3 molar equivalents relative to F6 or X6a;
iv) the reaction is conducted in an alcoholic solvent, preferably methanol; and
v) the temperature of the reaction is 30° C. to 70° C., preferably 40° C. to 50° C., more preferably 50° C.

The coupling reaction may be characterised by any one of i) to v) above. The coupling reaction may be characterised by any two of i) to v) above. The coupling reaction may be characterised by any three of i) to v) above. The coupling reaction may be characterised by any four of i) to v) above. The coupling reaction may be characterised by all of i) to v) above.

The coupling reaction may be characterised by i) and ii) above. The coupling reaction may be characterised by i) and iii) above. The coupling reaction may be characterised by i) and iv) above. The coupling reaction may be characterised by i) and v) above. The coupling reaction may be characterised by ii) and iii) above. The coupling reaction may be characterised by ii) and iv) above. The coupling reaction may be characterised by ii) and v) above. The coupling reaction may be characterised by iii) and iv) above. The coupling reaction may be characterised by iii) and v) above. The coupling reaction may be characterised by iv) and v) above.

In a preferred embodiment, a coupling reaction having excellent yield and minimal by-product formation is characterised by at least one of the following:
i) the catalyst is Pd(MeCN)₂Cl₂ in an amount of 0.25 mol % to 2 mol % relative to the number of moles of X6b, 0.25 mol % to 1.5 mol %, preferably 0.5 mol % or more preferably 1 mol % relative to the number of moles of X6b; (conversion of X6b to X6a is about 98%)
ii) the ligand is tBuPPh₂ in an amount of 0.5 mol % to 4% relative to the number of moles of X6b, preferably 1 mol % or 2 mol % relative to the number of moles of X6b; in particular the catalyst is Pd(MeCN)₂Cl₂, the ligand is tBuPPh₂ and the number of moles of tBuPPh₂ is twice the number of moles of Pd(MeCN)₂Cl₂;
iii) the base is KOH in an amount of 2 to 5 molar equivalents, preferably 3 molar equivalents relative to X6b;
iv) the reaction is conducted in MeTHF and water mixture; and
v) the temperature of the reaction is 30° C. to 70° C., preferably 60° C.

The coupling reaction may be characterised by any one of i) to v) above. The coupling reaction may be characterised by any two of i) to v) above. The coupling reaction may be characterised by any three of i) to v) above. The coupling reaction may be characterised by any four of i) to v) above. The coupling reaction may be characterised by all of i) to v) above.

The coupling reaction may be characterised by i) and ii) above. The coupling reaction may be characterised by i) and iii) above. The coupling reaction may be characterised by i) and iv) above. The coupling reaction may be characterised by i) and v) above. The coupling reaction may be characterised by ii) and iii) above. The coupling reaction may be characterised by ii) and iv) above. The coupling reaction may be characterised by ii) and v) above. The coupling reaction may be characterised by iii) and iv) above. The coupling reaction may be characterised by iii) and v) above. The coupling reaction may be characterised by iv) and v) above.

In a preferred embodiment, the borylation of X6b to X6a and the cross-coupling of X6a and F6 are conducted in a one-pot reaction.

### Preparation of X6b

X6b is a key intermediate in the novel synthesis described herein. Thus, the invention provides a synthetic intermediate, X6b:

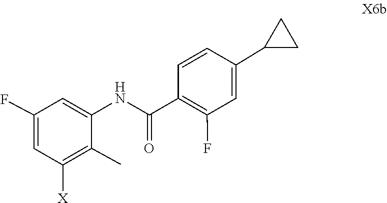

wherein X is F, Cl, Br, or I. Preferably, X is Br.

X6b can itself be synthesised in any suitable way. The invention provides methods of preparing synthetic intermediate X6b:

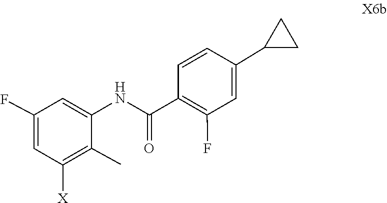

wherein X is F, Cl, Br, or I, preferably Br.

In some embodiments, the method comprises reacting compound X6d with compound N6a

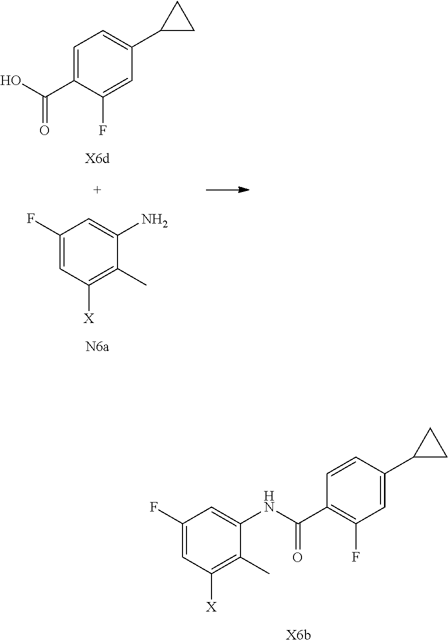

wherein X is Cl, Br, or I, preferably Br.

Carboxylic acid coupling reactions, including amidation reactions, are well-known to the skilled person, and typically involve reacting an amine with a carboxylic acid under coupling conditions, or converting the carboxylic acid group to an activated group that can react with an amine more easily.

Thus, in one embodiment, the synthesis of X6b involves using converting the carboxylic acid group of X6d into an activated carboxylic acid group. For example, the method can include conversion of compound X6d into compound X6c:

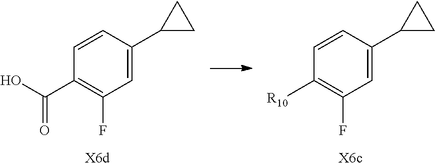

wherein R₁₀ is an activated carboxylic acid group, for example an acyl anhydride, acyl halide, or acyl phosphate, and wherein X is Cl, Br, or I. For example, conversion of X6d to the corresponding acyl chloride can be achieved using, thionyl chloride. The solvent may be an aromatic solvent such as toluene. The base may be pyridine. X6c can then be reacted with N6a to form compound X6b. These reactions can be conducted as a one-pot synthesis or sequentially. The formation of N6a from N6b may also be tied into this one-pot synthesis, such that X6c and N6a are prepared separately but then coupled

Alternatively, X6b is prepared directly from X6d and N6a by employing a carboxylic acid activating reagent. Carboxylic acid activating reagents are well known, and include HBT, HATU, HBTU, TBTU, HOBt, PyAOP, HCTU, PyClocK, TFFH, Carbodiimides (e.g. DCC), Carbonyl diimidazole (CDI), and Phosphonium salts (e.g. BOP, PyBOP).

The coupling of X6d or X6c and N6a can be conducted in the presence of a base, preferably a tertiary alkyl amine base such as triethylamine or DIPEA, or an aryl amine base such as pyridine. The coupling of X6d or X6c and N6a can be conducted in isopropylacetate, toluene, or preferably a mixture thereof.

X6d can be prepared from X6e:

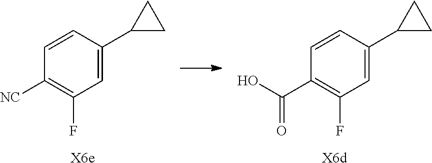

In one embodiment, X6d is prepared by contacting X6e with base, for example sodium hydroxide, which converts the cyano group to a carboxylic acid group.

X6e can be prepared from X6f:

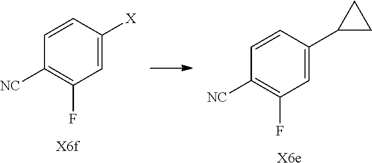

wherein X is Cl, Br, or I.

X6e is prepared by contacting X6f with X6g under cross-coupling conditions:

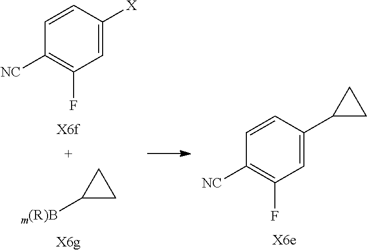

wherein X is F, Cl, Br, or I, m is 2 or 3 and R is F, Cl, Br, or I, OH, OC₁-C₆alkyl, N(C₁-C₆ alkyl)₂, aryl, or wherein two or three R groups other than F, Cl, Br, I, or OH can be taken together to form a cyclic boronate ester, for example pinacol boronate, or N-methyliminodiacetic acid (MIDA) boronate.

Coupling of organoboron and aryl halide compounds is described above in relation to the coupling of X6b and F7, and similar conditions can be used for the formation of X6e.

X6f can be prepared from X6h:

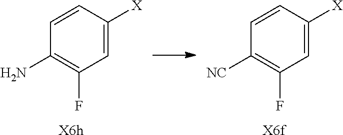

wherein X is Cl, Br, or I.

X6f can be prepared by diazotizing X6h, for example with nitrous acid or sodium nitrite under acidic conditions, followed by cyanation of the diazonium compound, for example using CuCN and/or NaCN.

X6 h can be prepared from X6i:

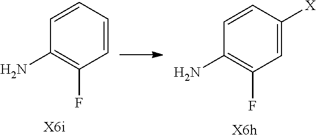

X6 h can be prepared by contacting X6i with a halogenating agent, for example a chlorinating agent such as AlCl₃, or N-chlorosuccinimide, a brominating agent selected from the group consisting of N-bromosuccinate, 1,3-Dibromo-5,5-Dimethylhydantoin (DBDMH), N-bromosuccinimide, TBAB, phosphorus tribromide, bromine chloride, aluminium tribromide, Br₂ and FeBr₃, HBr, tribromoisocyanuric acid, ammonium bromide with ozone, TBBDA, and combinations thereof, or an iodinating reagent such as N-iodosuccinimide. X6 h can also be prepared via a Sandmeyer reaction.

### Preparation of N6a

N6a is used in the preparation of X6b. N6a can be prepared from N6b:

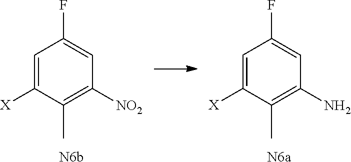

wherein Y is Cl, Br, or I.

N6a can be prepared by contacting N6b with a reducing agent, for example a reducing agent selected from the group consisting of: H₂ and Pt(V)/C; Raney nickel catalyst and H₂; Urushibara nickel catalyst and H₂; Adams' catalyst (PtO₂) and H₂; TiCl₃ and H₂; HCl and iron; NH₄Cl and iron; HCl and SnCl₂; samarium and NH₄Cl; FeCl₃, hydrazine hydrate; sodium hydrosulphite; hydrogen sulfide and base; hydroiodic acid; 1,3-dimethyl-2-imidazolidinone and sodium triethylsilanethiolate; and combinations thereof. In some embodiments, this reaction is conducted under micellar conditions.

N6b can be prepared from N6c:

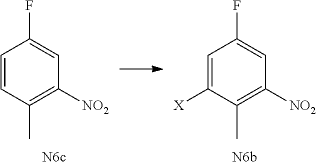

N6b can be prepared by contacting X6 h with a halogenating agent for example a chlorinating agent such as AlCl₃, or N-chlorosuccinimide, brominating agent selected from the group consisting of N-bromosuccinate, N-bromosuccinimide, 1,3-Dibromo-5,5-Dimethylhydantoin (DBDMH), TBAB, phosphorus tribromide, bromine chloride, aluminium tribromide, Br₂ and FeBr₃, HBr, tribromoisocyanuric acid, ammonium bromide with ozone, TBBDA, and combinations thereof, or an iodinating reagent such as N-iodosuccinimide. X6 h can also be prepared via a Sandmeyer reaction.

N6c can be prepared from N6d:

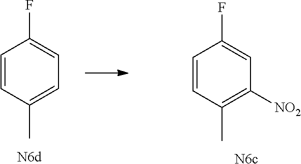

N6c can be prepared by contacting N6d with a nitrating agent, for example a nitrating agent selected from the group consisting of: nitric acid and sulfuric acid; nitric acid and acetic anhydride; tetrachloromethane, nitric acid and phosphorus pentoxide; isopentyl nitrate, trifluoromethanesulfonic acid, and 1-ethyl-3-methylimidazolium triflate; H-beta zeolite catalyst and N₂O₅; acetyl nitrate; and combinations thereof.

N6d can be prepared from N6e:

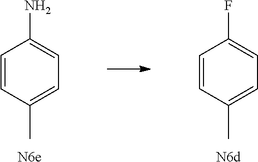

N6d can be prepared by contacting N6e with a diazotizing agent, such as nitrous acid or sodium nitrite under acidic conditions, followed by a fluorinating agent such as HF.

### Preparation of F6

F6 is used in the preparation of F7 and can itself be prepared by any suitable method. In one embodiment of the invention, F6 is prepared from F2 and F3:

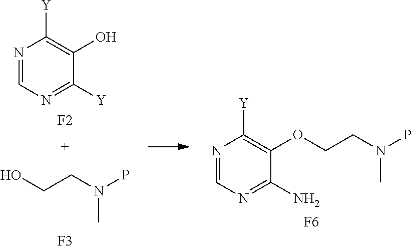

wherein Y is independently Cl, Br, or I.

In some embodiments, the preparation of F6 comprises reacting compound F2 with compound F3 to give compound F4:

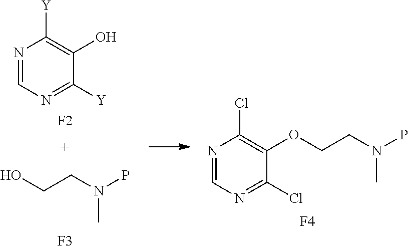

The reaction of F2 and F3 can be conducted under Mitsunobu conditions, for example in the presence of a phosphine compound such as PPh₃ (optionally on a resin support) and an azodicarbocylate such a DIAD or DEAD. In one embodiment, the reaction is conducted in an aromatic solvent such as toluene. In one embodiment, the solvent is dried to have a water content of less than 0.5 wt %, for example 0.1 wt %

The preparation of F6 may comprise converting F4 to F6:

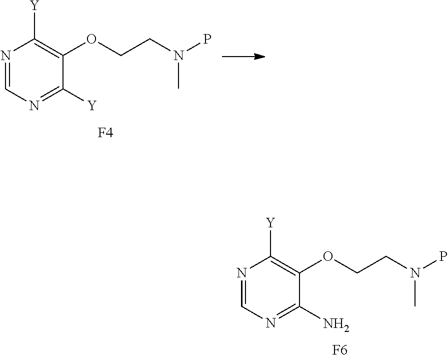

The conversion of F4 to F6 may be conducted using any suitable aminating reagent, for example ammonium hydroxide or water and ammonia. In one embodiment, the solvent is an alcoholic solvent such as iPrOH.

The reaction of F2 with F3 to give F4 and the conversion of F4 to compound F6 may be conducted in a sequential reaction or in a one-pot reaction.

Alternatively, F2 can be converted to F2′, via amination. Amination reagents include water and ammonia, or ammonium hydroxide, and this reaction may be conducted in a polar solvent such as an alcoholic solvent such as iPrOH. F2′ can then be reacted with F3, optionally under Mitsunobu conditions, for example in the presence of a phosphine compound such as PPh₃ and an azodicarbocylate such a DIAD or DEAD, to give F6:

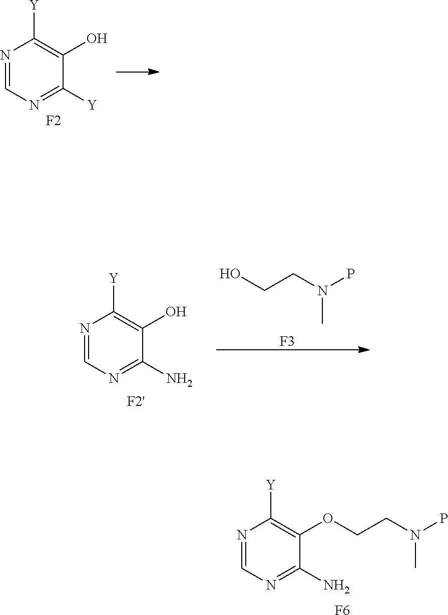

These reactions can be conducted in a sequential or in a one-pot fashion.

### Preparation of F11

Any of the reactions described herein can be used in the synthesis of compound F11:

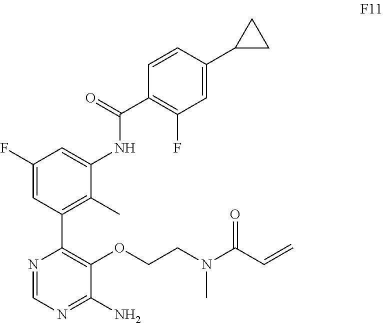 [SMILES: C=CC(=O)N(C)CCOc1c(N)ncnc1-c1cc(F)cc(NC(=O)c2ccc(C3CC3)cc2F)c1C]

In one embodiment of the methods of the invention, F7 is converted into F11 in one or more synthetic steps. For instance, in one embodiment, the methods of the invention may further comprise deprotection of F7 to give F8:

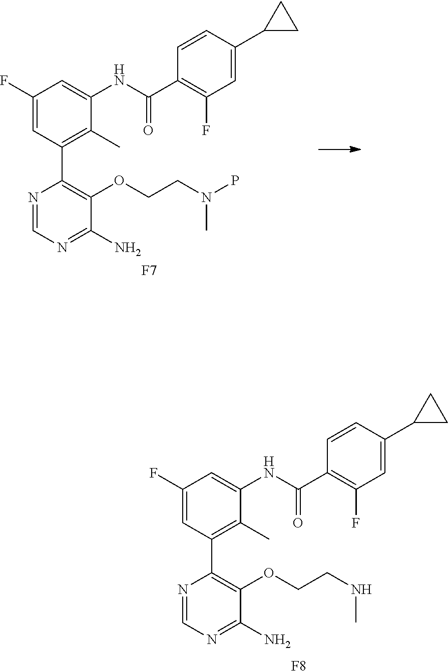

In some embodiments, P is a Boc group, and the deprotection is achieved using acid, for example HCl.

The methods of the invention may further comprise the conversion of F8 into F11:

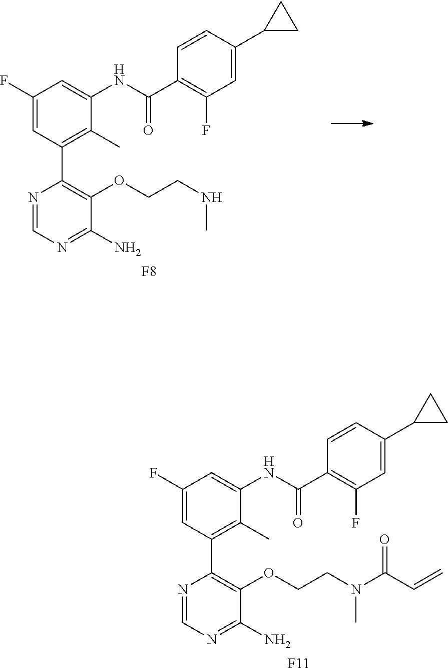

The conversion of F8 into F11 can be achieved by contacting F8 with F9:

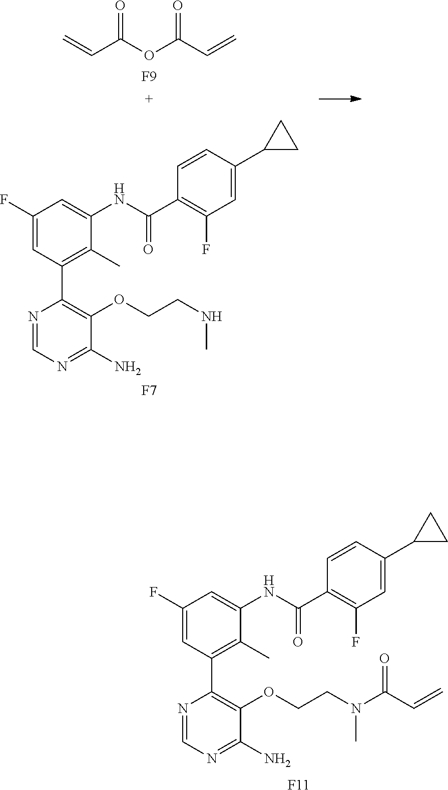

The formation of F11 from F8 and F9 can be achieved in the presence of base such as Na₂CO₃ and a suitable solvent, such as ethyl acetate. Alternatively, the reaction can be performed without base in a suitable solvent. In place of F9, acryloyl chloride can be used, or acrylic acid can be used in tandem with a carboxylic acid activating reagent, such as HBT, HATU, HBTU, TBTU, HOBt, PyAOP, HCTU, PyClocK, TFFH, Carbodiimides (e.g. DCC), Carbonyl diimidazole (CDI), or Phosphonium salts (e.g. T3P, SOCl₂ BOP, PyBOP). However, use of acrylic anhydride is preferred because it avoids the need for chromatography, unlike acrylic acid.

### Products Prepared According to Processes of the Invention and Uses Thereof

The invention provides synthetic routes to the compound remibrutinib. Hence, the protection afforded by patents arising from the present application may extends to the direct product of the processes herein, which is remibrutinib.

The invention provides compound F11 (remibrutinib), prepared or preparable by a process described herein. The synthesis of remibrutinib described herein does not involve INT 3 at any stage. Thus, in one embodiment, the remibrutinib prepared or preparable by a process described herein is substantially free from INT 3 (5-fluoro-2-methyl-3-(4,4,5,5-tetramethyl-1,3,2-dioxaborolan-2-yl)aniline). For example, the amount of INT 3 may be less than 100 ppm (parts per million), less than 10 ppm, less than 1 ppm, less than 100 ppb (parts per billion), less than 10 ppb, or less than 1 ppb. In one embodiment, the remibrutinib prepared or preparable by a process described herein contains no INT 3 (5-fluoro-2-methyl-3-(4,4,5,5-tetramethyl-1,3,2-dioxaborolan-2-yl)aniline). Alternatively or in addition, remibrutinib prepared or preparable by a process described herein is substantially free from (3-amino-5-fluoro-2-methylphenyl)boronic acid. For example, the amount of (3-amino-5-fluoro-2-methylphenyl)boronic acid may be less than 100 ppm (parts per million), less than 10 ppm, less than 1 ppm, less than 100 ppb (parts per billion), less than 10 ppb, or less than 1 ppb. In one embodiment, the remibrutinib prepared or preparable by a process described herein contains no 3-amino-5-fluoro-2-methylphenyl)boronic acid.

The invention also provides a pharmaceutical composition comprising remibrutinib prepared by or preparable by a process described herein, and thus may be substantially free from INT 3. In one embodiment, the composition also contains at least one pharmaceutically acceptable excipient, and often contains at least two or more pharmaceutically acceptable excipients. Some suitable excipients are disclosed herein. Other excipients may be used that are known in the art without departing from the intent and scope of the present application.

As used herein, the term “pharmaceutically acceptable excipients” includes any and all solvents, carriers, diluents, dispersion media, coatings, surfactants, antioxidants, preservatives (e.g., antibacterial agents, antifungal agents, antioxidants), isotonic agents, absorption delaying agents, salts, drug stabilizers, binders, additives, bulking agents, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). It should be understood that unless a conventional excipient is incompatible with the active ingredient, the use of any conventional excipient in any therapeutic or pharmaceutical compositions is contemplated by the present application.

The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, carriers or buffering agents, as well as adjuvants, such as solvents, preservatives, stabilizers, wetting agents, emulsifiers and bulking agents, etc.

Typically, the pharmaceutical compositions are tablets or capsules comprising the active ingredient together with at least one excipient, such as:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired;
d) carriers such as an aqueous vehicle containing a co-solvating material such as captisol, PEG, glycerin, cyclodextrin, or the like;
e) disintegrants, e.g., starches, agar, alginic acid or its sodium salt, or effervescent mixtures; and/or
f) absorbents, colorants, flavors and sweeteners.

Tablets may be either film coated or enteric coated according to methods known in the art. Preferably, the compound or composition is prepared for oral administration, such as a tablet or capsule, for example, and optionally packaged in a multi-dose format suitable for storing and/or dispensing unit doses of a pharmaceutical product. Examples of suitable packaging include, but are not limited to, hermetically sealed foils, unit dose containers (e. g., vials), blister packs, and strip packs.

Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The present invention further provides anhydrous pharmaceutical compositions and dosage forms comprising the remibrutinib prepared by or preparable by the methods described herein as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 10-50 mg of active ingredients. Preferably, the pharmaceutical composition or combination of the present invention can be in unit dosage of about 10 mg, about 25 mg or about 50 mg. The therapeutically effective dosage or amount of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg. Preferably, the therapeutically effective amount in vivo ranges between about 10 mg to about 200 mg daily, for example, about 10 mg, about 20 mg, about 25 mg, about 35 mg, about 50 mg, about 100 mg or about 200 mg daily. Preferably, the therapeutically effective amount in vivo is selected from about 10 mg, about 35 mg, about 50 mg or about 100 mg once a day. Also, preferably, the therapeutically effective amount in vivo is selected from about 10 mg, about 25 mg, about 50 mg or about 100 mg twice a day.

In another aspect, the present invention also provides a method for the treatment of disorders mediated by BTK or ameliorated by the inhibition of BTK, comprising administering to a patient in need of such treatment a therapeutically effective amount of remibrutinib prepared by or preparable by a method described herein.

In another aspect, the present invention also provides the use of remibrutinib prepared by or preparable by a method described herein for the preparation of a medicament for the treatment of disorders mediated by BTK or ameliorated by the inhibition of BTK.

In another aspect, the present invention also provides remibrutinib prepared by or preparable by a method described herein for use in the treatment of disorders mediated by BTK or ameliorated by the inhibition of BTK.

Remibrutinib prepared by or preparable by a method described herein is useful in the treatment of the following diseases or disorders mediated by BTK or ameliorated by inhibition of BTK: Autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenic purpura, systemic lupus erythematosus, multiple sclerosis, myasthenia gravis, Sjögren's syndrome, autoimmune hemolytic anemia, anti-neutrophil cytoplasmic antibodies (ANCA)-associated vasculitides, cryoglobulinemia, thrombotic thrombocytopenic purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerolunephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of haematopoietic origin including, but not limited to, multiple myeloma; a leukaemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenstroem disease.

Remibrutinib prepared by or preparable by a method described herein is especially useful in the treatment of rheumatoid arthritis; chronic urticaria, preferably chronic spontaneous urticaria; Sjögren's syndrome, multiple sclerosis, atopic dermatitis or asthma.

### EXAMPLES

The following non-limiting examples are illustrative of the disclosure.

An overview of the synthetic routes described herein and exemplified below is provided in the figures. The reactions are described in more detail below.

### Example 1—Preparation of F2

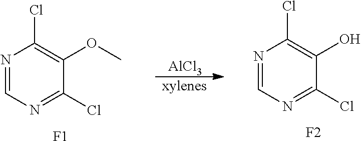

To a suspension of AlCl₃ in xylenes at 5° C. is added over 40 min a xylenes solution of F1. The mixture is warmed up to 30° C. over 60 min and stirred at this temperature overnight. EtOAc is added and the resulting solution is quenched over a 0.5 N aqueous solution of HCl at 0° C. for 1 h. The mixture is warmed to 25° C. and the phases are separated. The aqueous layer is discarded and the organic layer is concentrated. The resulting thin suspension is cooled to 20° C. at 0.3 K/min. The solids are filtered, the filter cake is washed with a 1:1 solution of xylenes and heptane and dried to afford F2 as a white solid in ca. 83% yield.

### Example 2—Preparation of F6

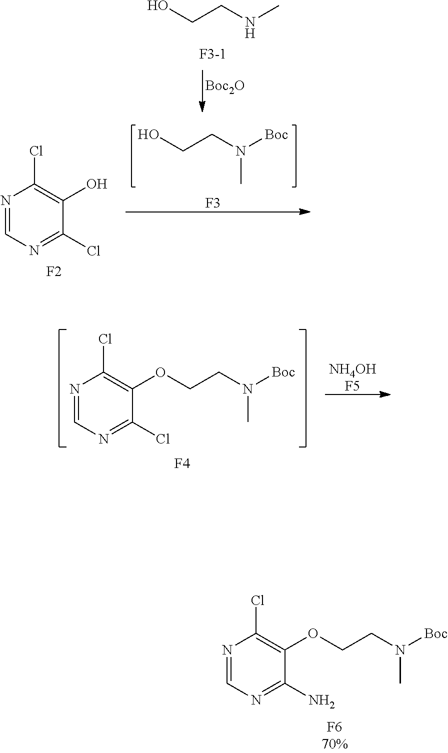

Preparation of the F3 solution: 13.0 g of water, 2.4 g of 30% sodium hydroxide solution, 68.0 g of toluene and 13.0 g of 2-methylaminoethanol are charged into reaction flask. Internal temperature is adjusted to 10-30° C. The reaction mixture is stirred for 25~35 min. Boc anhydride (37.8 g, 1.00 equiv) is added dropwise and the reaction mixture is stirred at 10~30° C. for another 6-12 hours. The reaction is quench with water (13.0 g) and the resulting biphasic mixture is stirred for 25-35 min. The lower water layer is removed and the organic layer is washed with another portion of water (13.0 g). The organic layer is used directly for the next step.

Mistunobu reaction to F4: A solution of F3 (1.4 eq) in toluene is dried by Dean Stark distillation to reach a water content of NMT 0.07 wt %. To the dried solution of F3 at 20-30° C. are added triphenylphosphine (42 g, 1.32 eq) and the reaction mixture is stirred at room temperature until a clear solution is observed. The reactor is inertized and cooled to ca. −30° C. F2 (20 g, 1.0 eq) is then added followed by DIAD (31.8 g, 1.30 eq) is added over 4 to 8 h maintaining the internal temperature between −25° C. The slightly turbid solution is warmed up to 10° C. within 4 h and stirred for another 15 to 20 h between 5 and 15° C. After completion of the reaction, toluene is distilled at 55° C. yielding a slightly viscous brownish-yellow suspension. The mixture is cooled to 10° C. and n-heptane (140 g) is added. The mixture is stirred for 2 h affording a light brown, well stirrable suspension. The suspension is filtered and the filter cake washed with cooled n-heptane. The filter cake containing triphenylphosphine oxide and H₂-DIAD is discarded. The combined mother and wash liquors are concentrated at JT 55° C. and 150 mbar to ca. ⅓ of their initial volume yielding a clear yellow solution of F4.

Amination to F6: The solvent of the solution of F4 is then switched to iPrOH via distillation and addition of iPrOH. To the yellow solution of F4 in iPrOH is added H₂O (3.5 w/w wrt F2) and 25 wt % NH₃ solution (3.5 w/w F2). The resulting yellow solution is stirred for 16 h at 70° C. A slight gas release (NH₃) is observed upon warming to 70° C. After completion of the reaction, the resulting yellow solution is cooled down to 45° C. over 40 min, and F6 seed crystals are added as a suspension in iPrOH. The suspension is aged for ca. 20 min. The thin suspension is then cooled to 10-20° C. at 10° C./h and aged for another 30 min. The suspension is filtered and the filter cake washed with a mixture (40 g) of H₂O and iPrOH (1:1). The wet product was dried at 50° C. under full vacuum (ca. 20 h) affording F6 as a white crystalline solid in ca. 70% yield.

### Example 3—Preparation of X6b

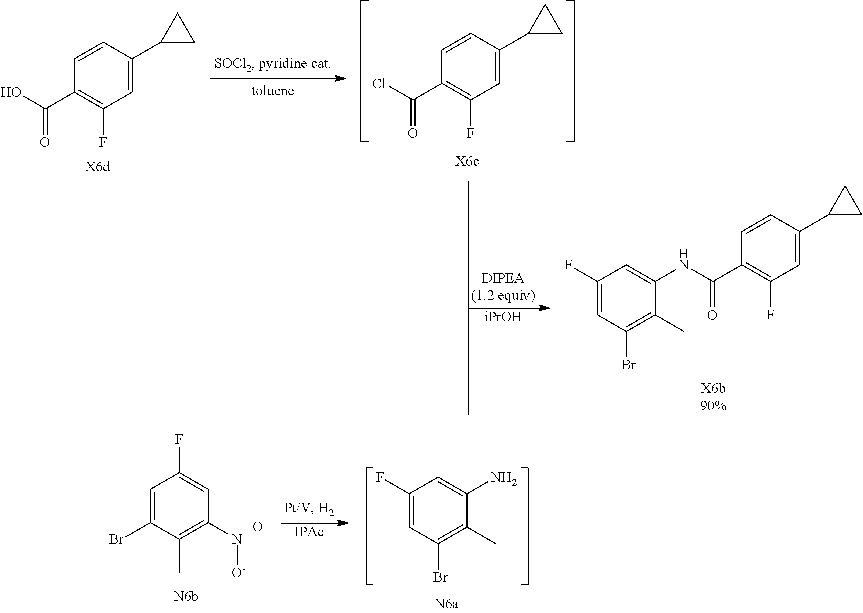

The synthesis of X6b is a highly convergent process that starts with the preparation of the N6a solution, the preparation of the acyl chloride X6c solution and the combination of the two solutions to form X6b.

Autoclave: preparation of the N6a solution: N6b (20 g, 1.0 equiv) is charged in an autoclave under N₂ and diluted with isopropylacetate (105 g). Then, ca. 1 wt % of wet Pt(V)/C (0.126 g dry weight) is added and the atmosphere is changed from N₂ to H₂. The hydrogenation is perfumed under 3 bars of H₂ for 12 hours with an internal temperature below 30° C. At the end of the reaction, the suspension is filtered to remove the catalyst. The reactor and the filter cake are rinsed with isopropylacetate. The N6a solution can be azeotropically distilled to remove the water or used as it is.

Reactor A: preparation of X6c solution: Under N₂ atmosphere, X6d (17 g, 1.1 equiv) is suspended in toluene (56 g). A catalytic amount of pyridine is added and the reaction mixture is heated to 50° C. Thionyl chloride is then added dropwise over 2 h and the resulting mixture is stirred for 1 at 50° C. The turbid solution is then distilled to half the volume, the reactor is refilled to its initial volume with toluene and the operation is repeated in order to remove the excess thionyl chloride. The X6c mixture is then cooled down to RT.

Reactor A: formation of X6b: To the solution of X6c (1.1 equiv) in toluene is added over 1 h the formerly prepared solution of N6a (1.0 equiv) in iPrOAc. At the end of the addition, DIPEA (13.4 g, 1.2 equiv) is carefully added over 2 h. The reaction mixture is stirred for 3 h after the end of the DIPEA addition and the reaction is quenched with iPrOH (26.4 g). The reaction is stirred overnight at RT and the suspension is filtered. The wet cake is rinsed with iPrOH and iPrOH/water. The cake is discharged and dried under reduced pressure. X6b is typically isolated in 87-93% yield.

### Example 4a: Optimization of Suzuki Condition for Conversion of X6a into F7

Previously, it was reported (DOI: 10.1021/acs.imedchem.9b01916) that the coupling reaction between F6 and X6a was carried out using 1 eq of F6, 1.15 eq of X6a, 5 mol % Pd(PPh₃)₂Cl₂, 3 eq of Na₂CO₃, 12 vol DME, 10 vol water at 75° C. for 8 h with a conversion of 74% isolated yield.

The cross-coupling reaction was optimized in order to replace the DME solvent with a class 3 solvent, suitable for commercial process, while also lowering the Pd loading and the production cost

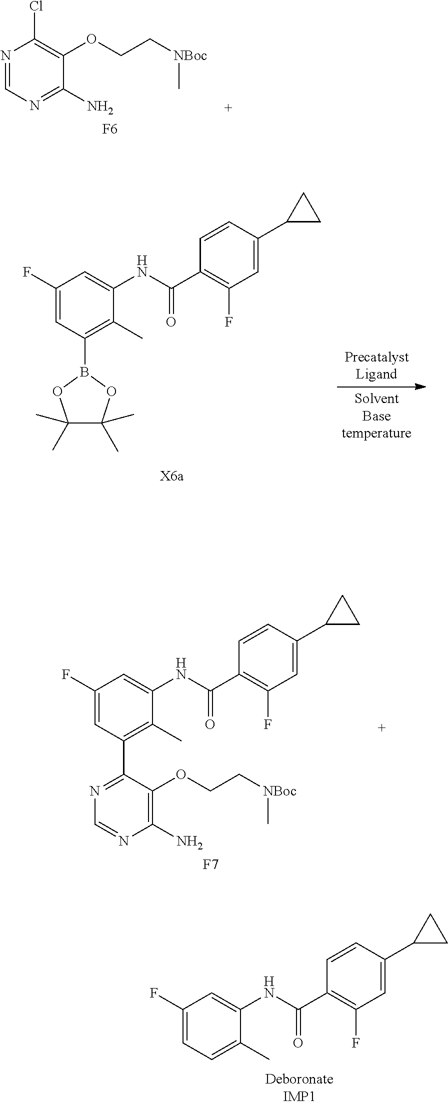

### Design and Experiment Details

1) Screened Suzuki 12 precatalysts and 6 solvent systems (80° C.: tert-Amyl alcohol, CPME and Toluene; 60° C.: THF, Me-THF and MeCN, combined with water respectively), using 1.15 eq. X6a at 2.0 mol % Pd level in the presence of 3.0 eq. K₃PO₄, after 16 h, found a series of precatalyst/solvent combinations that can promote reaction with full conversion, De-Boronate is the major side-product; decided to carry out full ligand screening in both Toluene (80° C.) and Me-THF (60° C.)
2) Screened 48 ligands in 10.0 vol. Me-THF/3.0 vol. water at 60° C. or in 10.0 vol. Toluene/3.0 vol. water at 80° C., using 2.0 mol % Pd(OAc)₂, 1.1 eq. Boronate and 3.0 eq. K₃PO₄, after 16 h, found 5 ligands (RuPhos, dppf, S-Phos, Cy₃P·HBF₄ and Ph₂P(t-Bu)) can promote reactions with full conversion with leading Prod/IS in Me-THF/water at 60° C., and De-Boronate side-product can be controlled at 3% to 8% level

| | | | | | De-Boronate/ |
|---|---|---|---|---|---|
| Solvent | Ligand | Temperature | Base | Conversion | Prod |
| Toluene | Cy₃P—HBF₄ | 80 degree | K₃PO₄ | 97% | 17% |
| Me—THF | Cy₃P—HBF₄ | 60 degree | K₃PO₄ | 100% | 4% |
| Me—THF | RuPHOS | 60 degree | K₃PO₄ | 100% | 4% |
| Me—THF | S—PHOS | 60 degree | K₃PO₄ | 100% | 8% |
| Me—THF | Ph₂P(tBu) | 60 degree | K₃PO₄ | 100% | 3% |
| Me—THF | Cy-BIPHEP | 60 degree | K₃PO₄ | 99% | 8% |
| Me—THF | SPHOS——SO₃Na | 60 degree | K₃PO₄ | 99% | 6% |
| Me—THF | dppf | 60 degree | K₃PO₄ | 100% | 3% |

3) Keep P:Pd at 2:1 ratio, screened 6 Pd precursors (Pd(OAc)₂, [Pd(C₃H₅)Cl]₂, Pd(TFA)₂, Pd(MeCN)₂Cl₂, Pd₂(dba)₃ and PdBr₂) at 1.0 mol % Pd level, combined with RuPhos, dppf, S-Phos, Cy₃P·HBF₄ and Ph₂P(t-Bu) respectively, in the presence of 3.0 eq. K₃PO₄ and 1.05 eq. X6a in 10.0 vol. Me-THF/3.0 vol. water at 60° C., after 16 h, still keep Cy₃P·HBF₄ and Ph₂P(t-Bu) as optimal ligand candidates, meanwhile, Pd(TFA)₂, Pd(MeCN)₂Cl₂ and PdBr₂ as leading Pd precursors

| | | | | | | De- |
|---|---|---|---|---|---|---|
| Solvent | Ligand | Temperature | Pd Precursor | Base | Conversion | Boronate/Prod |
| Me—THF | Ph₂P(t-Bu) | 60 degree | Pd(MeCN)₂Cl₂ | K₃PO₄ | 100% | 2% |
| Me—THF | RuPhos | 60 degree | Pd(MeCN)₂Cl₂ | K₃PO₄ | 100% | 2% |
| Me—THF | Ph₂P(t-Bu) | 60 degree | Pd(TFA)₂ | K₃PO₄ | 100% | 1% |
| Me—THF | Ph₂P(t-Bu) | 60 degree | PdBr₂ | K₃PO₄ | 100% | 2% |
| Me—THF | Cy₃P·HBF₄ | 60 degree | Pd(TFA)₂ | K₃PO₄ | 100% | 3% |

4) Keep P:Pd at 2:1 ratio, using Cy₃P·HBF₄ and/or Ph₂P(t-Bu) as ligand, combined with Pd(TFA)₂, Pd(MeCN)₂Cl₂ and PdBr₂ respectively, screened Pd loading from 0.1 to 2.0 mol % in the presence of 3.0 eq. K₃PO₄ and 1.05 eq. X6a in 10.0 vol. Me-THF/3.0 vol. water at 60° C., after 16 h, found Pd(MeCN)₂Cl₂/Ph₂P(t-Bu) is the leading optimal precatalyst combination, and Pd loading can be dropped to 0.3 to 0.5 mol %, De-Boronate/Prod can be controlled at around 1%

| | | | | | Precatalyst | | De-Boronate/ |
|---|---|---|---|---|---|---|---|
| Solvent | Ligand | Temperature | Pd Precursor | Base | Loading | Conversion | Prod |
| Me—THF | Ph₂P(t-Bu) | 60 degree | Pd(TFA)₂ | K₃PO₄ | 0.8 mol % | 100% | 2% |
| Me—THF | Ph₂P(t-Bu) | 60 degree | Pd(MeCN)₂Cl₂ | K₃PO₄ | 0.8 mol % | 100% | 2% |
| Me—THF | Ph₂P(t-Bu) | 60 degree | Pd(MeCN)₂Cl₂ | K₃PO₄ | 0.5 mol % | 100% | 1% |
| Me—THF | Ph₂P(t-Bu) | 60 degree | Pd(TFA)₂ | K₃PO₄ | 1.0 mol % | 100% | 2% |
| Me—THF | Ph₂P(t-Bu) | 60 degree | Pd(TFA)₂ | K₃PO₄ | 1.5 mol % | 100% | 2% |
| Me—THF | Ph₂P(t-Bu) | 60 degree | Pd(MeCN)₂Cl₂ | K₃PO₄ | 0.3 mol % | 99% | 1% |
| Me—THF | Ph₂P(t-Bu) | 60 degree | Pd(MeCN)₂Cl₂ | K₃PO₄ | 1.5 mol % | 100% | 2% |
| Me—THF | Ph₂P(t-Bu) | 60 degree | PdBr₂ | K₃PO₄ | 1.0 mol % | 100% | 2% |
| Me—THF | Ph₂P(t-Bu) | 60 degree | Pd(MeCN)₂Cl₂ | K₃PO₄ | 2.0 mol % | 100% | 2% |
| Me—THF | Ph₂P(t-Bu) | 60 degree | PdBr₂ | K₃PO₄ | 0.5 mol % | 100% | 1% |
| Me—THF | Cy₃P·HBF₄ | 60 degree | Pd(MeCN)₂Cl₂ | K₃PO₄ | 1.0 mol % | 99% | 3% |

5) Using Pd(MeCN)₂Cl₂/Ph₂P(t-Bu) as the optimal precatalyst combination and 1.05 eq. X6a, screened Pd loading from 0.1 to 0.5 mol % in the presence of K₂CO₃, Cs₂CO₃, K₃PO₄ and KF respectively, found K₃PO₄ is the optimal base, 0.3 to 0.5 mol % Pd(MeCN)₂Cl₂/Ph₂P(t-Bu) precatalyst is suggested in scaled-up reaction

### Best Conditions

1) 1.0 eq. F6 1.05 eq. X6a, 0.5 mol % Pd(MeCN)₂Cl₂, 1.0 mol % Ph₂P(t-Bu), 3.0 eq. K₃PO₄ in 10.0 vol. Me-THF/3.0 vol. water at 60° C. for 16 h, reaction achieved a full conversion with 90.6% H PLC IPC purity, 1% De-Boronate/Prod
2) 1.0 eq. F6, 1.05 eq. X6a, 0.3 mol % Pd(MeCN)₂Cl₂, 0.6 mol % Ph₂P(t-Bu), 3.0 eq. K₃PO₄ in 10.0 vol. Me-THF/3.0 vol. water at 60° C. for 16 h, reaction achieved 99% conversion with 88.5% HPLC IPC purity, 1% De-Boronate/Prod

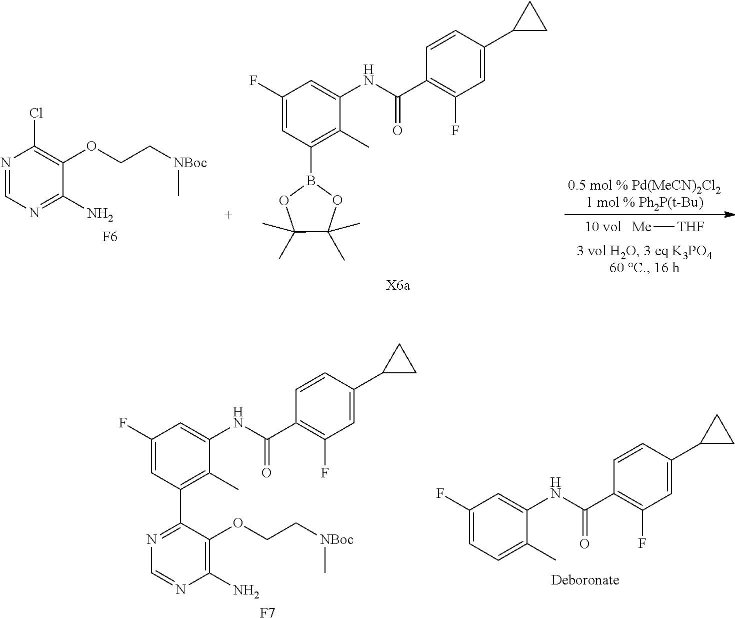

### Next Optimal Conditions

1) 1.0 eq. F6, 1.05 eq. X6a, 0.8 mol % Pd(TFA)₂, 1.6 mol % Ph₂P(t-Bu), 3.0 eq. K₃PO₄ in 10.0 vol. Me-THF/3.0 vol. water at 60° C. for 16 h, reaction achieved a full conversion with 90.9% H PLC IPC purity, 2% De-Boronate/Prod
2) 1.0 eq. F6, 1.05 eq. X6a, 0.8 mol % Pd(MeCN)₂Cl₂, 1.6 mol % Ph₂P(t-Bu), 3.0 eq. K₃PO₄ in 10.0 vol. Me-THF/3.0 vol. water at 60° C. for 16 h, reaction achieved a full conversion with 91.2% HPLC IPC purity, 2% De-Boronate/Prod

### Example 4b—Preparation of F7 One Pot-Borylation-Suzuki Cross Coupling from X6b Using Optimized Condition from Example 4a

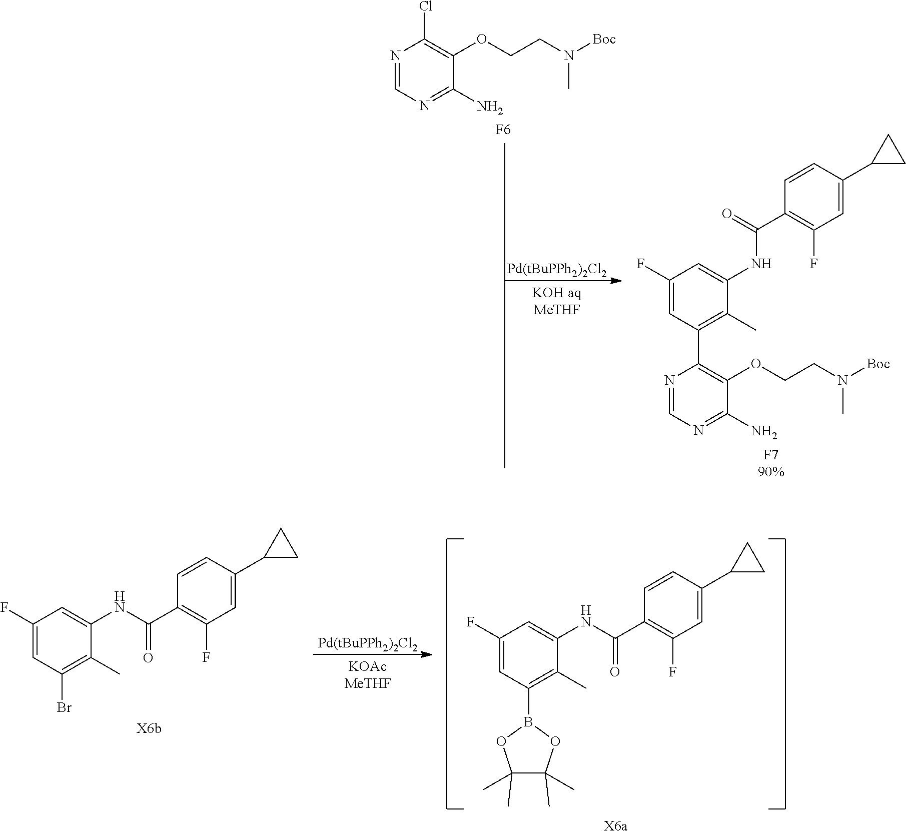

Miyaura borylation: X6b (1.0 equiv), B₂pin₂ (1.06 equiv) and KOAc (2.5 equiv) are charged in a reactor under N₂ atmosphere containing degazed Me-THF. Water content of the reaction mixture is measured and adjusted between 1000 and 2500 ppm. After inertisation of the vessel, a solution of Pd(MeCN)₂Cl₂ (0.5 mol %) in degazed MeTHF and a solution of PPh₂tBu (1.0 mol %) in degazed MeTHF are successively added. The reaction mixture is then heated to 70° C. for 16 h.

Suzuki coupling: Once full conversion of X6b is achieved (X6b<0.25%, conversion is about 98%), the reaction mixture is cooled to RT and the reaction mixture is quenched with an aqueous solution of KOH (21% wt/wt). The aqueous layer is separated and discarded and a fresh portion of aqueous solution of KOH (21% wt/wt) is added. F6 (0.96 equiv compared to X6b) is added as a solid followed by, after appropriate degazing, a second portion of PPh₂tBu (2 mol %) in degazed MeTHF and a second portion of Pd(MeCN)₂Cl₂ (1 mol %) in degazed MeTHF. The reaction mixture is then heated to 60° C. for ca. 24 h. After completion of the reaction, an aqueous solution of N-acetyl cysteine is added to the reaction mixture at 60° C. After stirring for 2 h, the aqueous layer is discarded. Another portion of aqueous N-acetyl cysteine solution is added and the pH is adjusted 9.5 by addition of aq. solution of KOH. After stirring for 2 h, the aqueous layer is discarded. The organic layer is then washed with water for 30 min and the aqueous layer is discarded. The solution is filtered at 60° C. over active charcoal and the solution is concentrated to half its volume by distillation under reduced pressure. n-Heptane is slowly added and the resulting suspension is cooled to 20° C., stirred for 2 h and filtered. The filter cake is washed with a mixture of 1:5 Me-THF and n-heptane. In case the purity is not satisfactory, the wet cake can be reslurried in Me-THF and n-heptane (1:5). The cake is discharged and dried under reduced pressure. F7 is typically isolated in 92% yield.

### Example 4c—Development of a One-Pot Borylation/Suzuki Cross-Coupling Using Tetrahydroxydiboron to Use in the Preparation of F7

A one-pot borylation/Suzuki cross-coupling using tetrahydroxydiboron has been developed for the synthesis of F7 from X6b using BBA as borylating reagent. This process is characterized by the utilization of remarkably low loadings of Pd-catalyst, the avoidance of pinacol hydrate precipitates in the final product and the use of methanol as a green alcoholic solvent over both steps. This process addresses some of the previous problems associated with the use of bis(pinacolato) diboron as borylating reagent, thus becoming a more atom efficient and cost-effective approach. Preliminary results demonstrated the feasibility of this one-pot process in a 2.2 g scale using a FlexyALR reactor.

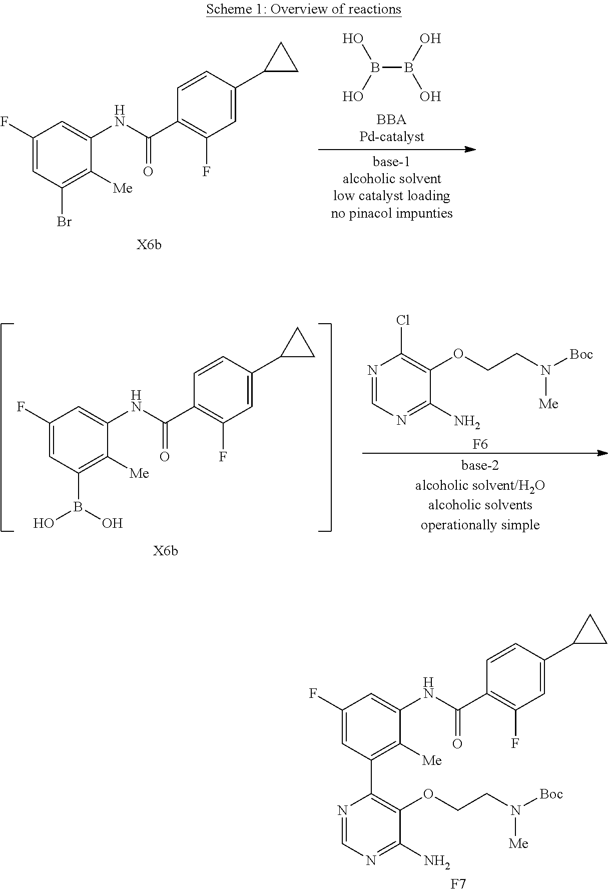

### Results and Discussion

Miyaura Borylation: In order to develop optimal reaction conditions for the Miyaura borylation of using BBA, we screened crucial reaction parameters such as the catalytic system, base, solvent and temperature. This borylation was restricted to the utilization of Pd(II)-precatalysts which promote fast Pd(0) formation. Indeed, the utilization of 2nd generation Buchwald precatalysts in combination with two equivalents of additional ligand, proved to be the best catalytic system in our reaction (Table 1, entries 1-6). Out of all the screened precatalysts, only Pd-XPhos-2G afforded full conversion of the starting material while providing the highest yield and selectivity towards the formation of X6a (entry 2). In a similar manner, the utilization of ethylene glycol as additive also proved to be highly beneficial, as full conversion could not be achieved without it (entry 1 vs 2). BBA can be in situ stabilized through the formation of the corresponding boronic ester derivative, allowing the reduction in the amount of borylating reagent and Pd while increasing the rate of the borylation. A further reduction of the catalyst loading was attempted (entries 8-10). Surprisingly, reducing the catalyst loading afforded lower amounts of reduce and dimerized products IMP1 and IMP2, while still affording almost full conversion of X6b (entry 8). Additionally, higher conversion was observed by increasing the reaction time, thus suggesting that BBA was still present in the reaction mixture (entry 9). These results could indicate the formed boronic acid might undergo Pd(II)-catalyzed decomposition pathways, and that higher amount of Pd source in the presence of trace amount of oxygen might favor this pathway. Finally, simply by increasing the reaction temperature to 50° C., full conversion to the final product was observed in high selectivity and yield (entry 10).

TABLE 1
Screening results from the Miyaura borylation using BBA and KOAc.

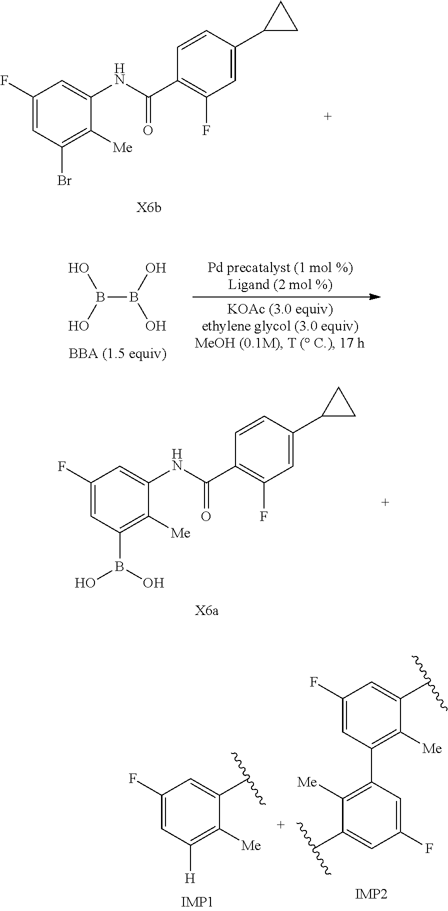 [SMILES: Cc1c(Br)cc(F)cc1NC(=O)c1ccc(C2CC2)cc1F ; OB(O)B(O)O ; Cc1c(NC(=O)c2ccc(C3CC3)cc2F)cc(F)cc1B(O)O ; *c1cc(F)ccc1C ; *c1cc(F)cc(-c2cc(F)cc(*)c2C)c1C]

| Entry^{a} | Pd-precatalyst | T (° C.) | X6b (%)^{a} | X6a (%)^{a} | IMP1 (%)^{a} | IMP2 (%)^{a} |
|---|---|---|---|---|---|---|
| 1 | XPhos-2G | 40 | 4 | 76 | 9 | 8 |
| 2 | XPhos-2G | 40 | 0 | 87 | 8 | 5 |
| 3 | Aphos-2G | 40 | 16 | 38 | 16 | 28 |
| 4 | RuPhos-2G | 40 | 43 | 39 | 11 | 4 |
| 5 | SPhos-2G | 40 | 50 | 36 | 7 | 3 |
| 6 | cataCXium-3G | 40 | 27 | 37 | 29 | 3 |
| 7 | Pd(PCy₃)₂ | 40 | 54 | 26 | 14 | 4 |
| 8 | XPhos-2G (0.25%) | 40 | 5.8 | 88.5 | 4.6 | 1.0 |
| 9 | XPhos-2G (0.25%) ^{b} | 40 | 3.7 | 90.9 | 4.3 | 1.1 |
| 10 | XPhos-2G (0.25%) | 50 | 0 | 94.9 | 4.6 | 0.5 |

Reaction conditions: X6b (1.0 equiv), BBA (1.5 equiv), KOAc (3.0 equiv), ethylene glycol (3.0 equiv), Pd-precatalyst (1 mol %), Ligand (2 mol %), MeOH (0.1 M), T (° C.), 17 h.
^{a}Liquid Chromatography Area Percent of compound (LCAP).
^{b} Reaction time 20 h.

We also decided to evaluate the reaction replacing the ethylene glycol with using an amine base, DIPEA, and other Buchwald precatalysts to see if we could further improve the results for the Miyaura Borylation and increase the amount of working catalysts (Table 2, entries 1-5). Although most catalysts performed worse under these conditions, a hit was found by using Pd-cataCXium 3G (entry 5). Although slightly higher amount of IMP1 and IMP2 were formed as compared to the previously optimized conditions, these results were promising considering cataCXium outperformed XPhos when used in combination with DIPEA (entry 5 vs 1). Having this result in hand, we screened some critical reaction parameters to see if this result could be further improved (entries 6-9). Considering our previous results, a reduction of the catalyst loading was first examined (entry 6). Importantly, we discovered that 0.05 mol % Pd was enough to drive the reaction to completion, suggesting that the catalytic activity of Pd-cataCxium-3G under these conditions was much higher than that of Pd-XPhos-2G. Importantly, heating to 50° C. was found optimal, as decreasing the temperature resulted in incomplete reactions (entry 7). Surprisingly, we discovered that the addition of ethylene glycol was detrimental for the conversion of the reaction, thus suggesting that cyclic diboron species might be less reactive under these conditions (entry 8). Although a remarkably high catalytic activity was seen under these newly optimized conditions, the relative amounts of byproducts IMP1 and IMP2 could not be further decreased, and the conditions based on the utilization of Pd-Xphos-2G, KOAc and ethylene glycol remained superior.

TABLE 2
Screening results from the Miyaura borylation using BBA and DIPEA.

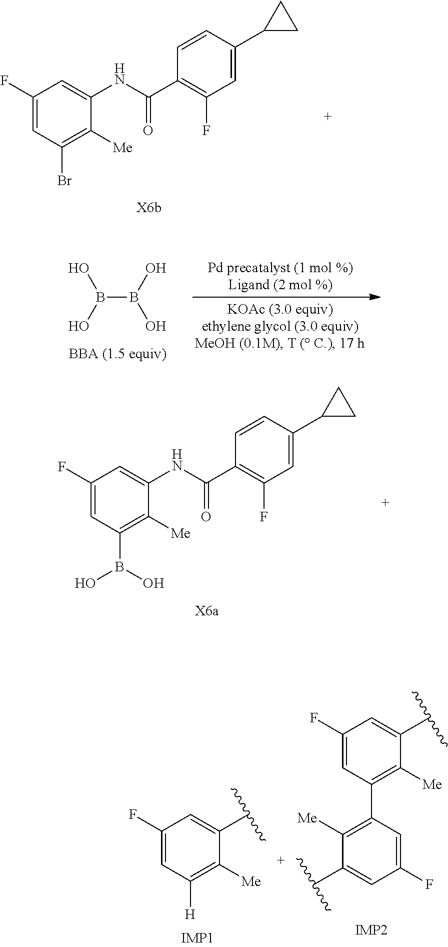 [SMILES: Cc1c(Br)cc(F)cc1NC(=O)c1ccc(C2CC2)cc1F ; OB(O)B(O)O ; Cc1c(NC(=O)c2ccc(C3CC3)cc2F)cc(F)cc1B(O)O ; *c1cc(F)ccc1C ; *c1cc(F)cc(-c2cc(F)cc(*)c2C)c1C]

| Entry | Pd-precatalyst | T (° C.) | X6b ( %)^{a} | X6a (%)^{a} | IMP1 (%)^{a} | IMP2 |
|---|---|---|---|---|---|---|
| 1 | Xphos-2G | 50 | 26 | 61 | 9 | 4 |
| 2 | Sphos-2G | 50 | 39 | 52 | 6 | 4 |
| 3 | PAphos-2G | 50 | 23 | 65 | 6 | 6 |
| 4 | PCy3-2G | 50 | 57 | 30 | 7 | 6 |
| 5 | cataCXium-3G | 50 | 0 | 87 | 7 | 6 |
| 6 | cataCXium-3G (0.05%) | 50 | 0 | 86 | 8 | 6 |
| 7 | cataCXium-3G (0.05%) | rt | 23 | 68 | 6 | 3 |
| 8 | cataCXium-3G ^{b} | rt | 69 | 20 | 11 | 1 |

Reaction conditions: X6b (1.0 equiv), BBA (1.5 equiv), DIPEA (3.0 equiv), Pd-precatalyst (0.25 mol %), Ligand (0.5 mol %), MeOH (0.1 M), T (° C.), 17 h.
^{a}Liquid Chromatography Area Percent of compound (LCAP).
^{b} Ethylene glycol (3.0 equiv) was added to the reaction mixture.

Suzuki Cross-Coupling: Having in hand two sets of optimized conditions for the synthesis of boronic acid X6a by using BBA as borylating reagent, we studied the viability of the subsequent Suzuki coupling with the ultimate goal of developing a one-pot process for the synthesis of F7. For this purpose, we initially attempted the Suzuki-coupling of X6a and F6 under Molanders previously developed (Gurung, S. R., et al., Org. Process Res. Dev. 2017, 21, 65-74) reaction conditions at 60° C. (Table 3, entry 1). Disappointingly however, uneven and incomplete conversion of X6a and F6 was observed after heating to 60° C. for 17 hours. Additionally, we found F6 partially reacted through an S_{N}Ar pathway with EtOH to form the corresponding ether. At this point, we wondered whether the utilization of milder organic bases such as amines could help diminish this side reaction. Indeed, the utilization of Et₃N resulted in minimal formation of the C-0 coupling product, leading to an even and almost complete conversion of X6a and F6 (entry 2). Finally, we were surprised to find that MeOH was superior to EtOH providing full conversion of X6a and F6, and higher yields of the coupled product (entry 3). Additionally, F7 precipitated out directly from the reaction mixture, thus simplifying significantly the final workup purification. Although the formation of reduced and dimerized products IMP1 and IMP2 evidenced the presence of trace amount of oxygen in the reaction solvent, we expected that the scale up the process should effectively eliminate this problem (entries 1-3).

TABLE 3
Screening results from the Suzuki coupling with F6.

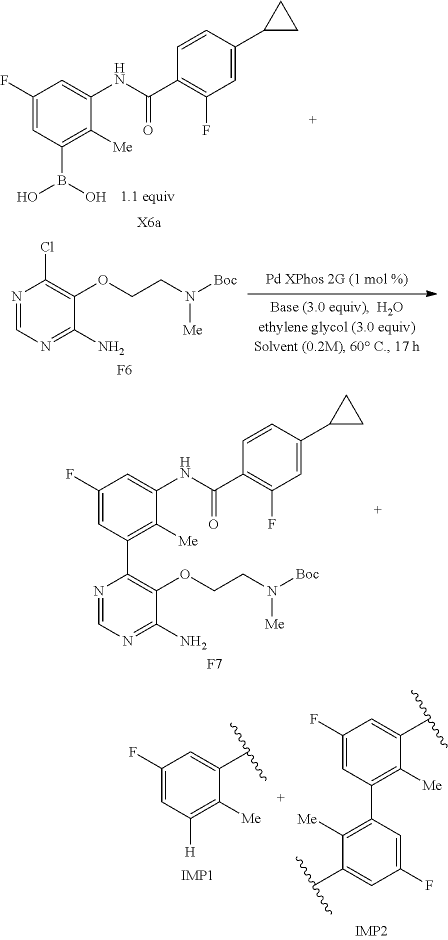

| Entry | Base | Solvent | X6a (%)^{a} | F6 (%)^{a} | F7 (%)^{a} | IMP1 (%)^{a} | IMP2 (%)^{a} |
|---|---|---|---|---|---|---|---|
| 1 | K₃PO₄ | EtOH | 43 | 15 | 28 | 8 | 5 |
| 2 | Et₃N | EtOH | 0 | 5 | 67 | 20 | 9 |
| 3 | Et₃N | MeOH | 0 | 0 | 77 | 7 | 13 |

Reaction conditions: X6a (1.1 equiv), F6 (1.0 equiv), Base (3.0 equiv), ethylene glycol (3.0 equiv), Pd-XPhos 2G (1.0 mol %), Solvent (0.2 M), 60 °C., 17 h.
^{a}Liquid Chromatography Area Percent of compound (LCAP).

One-pot borylation and coupling: As Pd-XPhos 2G and Pd-cataCXium 3G were the leading precatalysts in the Miyaura borylation using BBA, we decided to compare the efficiency of these two catalysts using our optimized conditions in a one-pot process (Table 4). As shown in entry 1, Pd-XPhos 2G demonstrated to be superior to Pd-cataCXium 3G in the one-pot procedure, affording a 79% isolated yield of F7 with a 78% purity starting from X6b. As expected, the workup and purification of F7 could performed through direct filtration and washing of the formed precipitate with a MeOH/H₂O mixture.

TABLE 4
Screening results from the one-pot reaction.

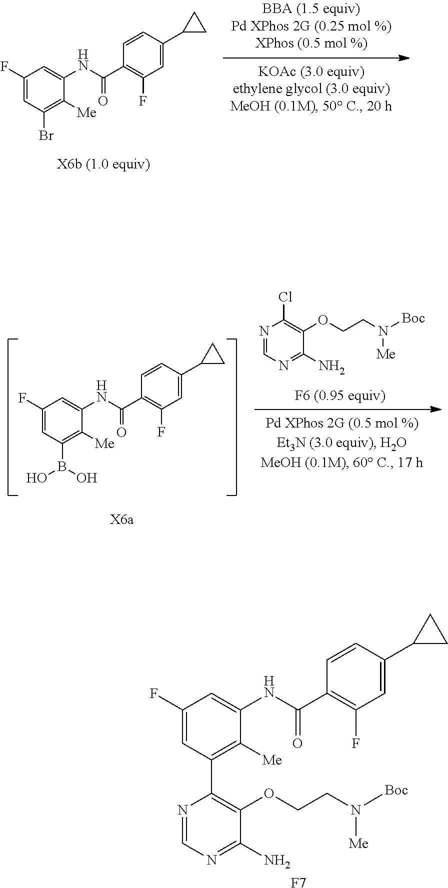

| Entry | Pd-Precatalyst | X6a (%)^{a} | F6 (%)^{a} | F7 (%)^{a} | IMP1 (%)^{a} | IMP2 (%)^{a} |
|---|---|---|---|---|---|---|
| 1 | XPhos 2G | 5 | 1 | 74 (79%) | 8 | 8 |
| 2 | cataCXium 3G | 13 | 4 | 61 | 11 | 6 |

Miyaura borylation: Reaction conditions: X6b (1.0 equiv), BBA (1.5 equiv), KOAc (3.0 equiv), ethylene glycol (3.0 equiv), Pd-XPhos 2G (0.25 mol %), XPhos (0.5 mol %), MeOH (0.1 M), 50 °C., 17 h. Suzuki-coupling: X6a (1.0 equiv), F6 (0.95 equiv), Et₃N (3.0 equiv), Pd-XPhos 2G (1.0 mol %), MeOH (0.1 M), 50° C., 17 h.
^{a}Liquid Chromatography Area Percent of compound (LCAP).

Scale-up: Having developed conditions for both steps in MeOH, using the same precatalyst under mild conditions, the one-pot reaction was attempted on a larger scale (2.2 g of X6b) using a Flexy ALR-1 300 ml reactor (Table 5).

X6b (2.20 g, 1.0 equiv.), potassium acetate (1.76 g, 3.0 equiv.), ethylene glycol (1.0 ml, 3.0 equiv.) and MeOH (100 ml) were charged in a 300 ml FlexyALR reactor. The reaction mixture was degassed through successive vacuum/N₂ cycles and a solid mixture of BBA (807 mg, 1.5 equiv.), Pd XPhos 2G (12 mg, 0.25 mol %) and XPhos (14 mg, 0.50 mol %) was added under N₂. After degassing a second time, the reaction was heated to 50° C. and stirred overnight. The mixture containing the boronic acid was then cooled to 20° C., and F6 (1.73 g, 0.95 equiv.), Pd XPhos 2G (24 mg, 0.5 mol %), Et₃N (2.5 ml) and degassed water (30 ml) were added under N₂. The reaction was degassed a third time and stirred at 60° C. overnight. Subsequently, it was cooled to 40° C. and concentrated under reduced pressure (ca. 40 ml MeOH removed). The reaction mixture was then cooled to 20° C. and stirred for 3 hours. The light brown suspension was filtered off, washed with a cold solution of MeOH/H₂O 4/1 (40 ml) and dried to afford F7 (1.87 g, 56%) as a brown solid.

TABLE 5
Scale up of the one-pot process.

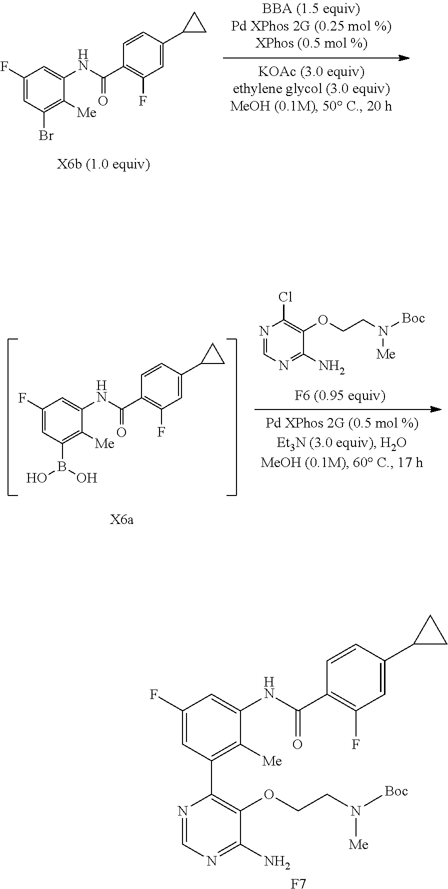

| Step | X6b (%)^{a} | X6a (%)^{a} | IMP1 (%)^{a} | IMP2 (%)^{a} | F6 (%)^{a} | F7 (%)^{a} |
|---|---|---|---|---|---|---|
| Borylation | 0 | 93.9 | 4.6 | 1.4 | | |
| Suzuki | | 21 | 7 | 1 | 3 | 63 (56) |

Miyaura borylation: Reaction conditions: X6b (1.0 equiv), BBA (1.5 equiv), KOAc (3.0 equiv), ethylene glycol (3.0 equiv), Pd-XPhos 2G (0.25 mol %), XPhos (0.5 mol %), MeOH (0.1 M), 50° C., 17 h. Suzuki-coupling: X6a (1.0 equiv), F6 (0.95 equiv), Et₃N (3.0 equiv), Pd-XPhos 2G (0.5 mol %), MeOH (0.1 M), 60° C., 17 h.
^{a}Liquid Chromatography Area Percent of compound (LCAP).

We were glad to find out that the Miyaura borylation of X6b worked well affording the desired intermediate X6a in excellent yield and selectivity. Interestingly, as described by Molander for the utilization of Pd-XPhos 2G the end of the borylation was evidenced through the sudden color change of the reaction mixture from white to light orange. Subsequently, the Suzuki-coupling was carried out adding F6, a new batch of catalyst, Et₃N and H₂O to the reaction mixture. Filtration and washing of the final product afforded F7 in a 56% isolated yield over both steps with an 87% IPC purity. Importantly, as we had anticipated, excluding all traces of oxygen by carrying out both steps in a reactor minimized the formation of byproduct IMP1 and IMP2.

### Example 5—Preparation of F8

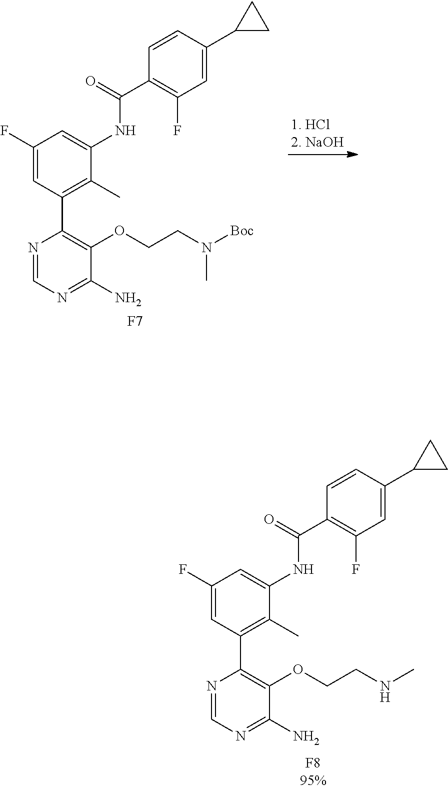

F7 is suspended in isopropyl acetate at 25° C. and concentrated hydrochloric acid (approx. 37% w/w, 4.1 equiv) is dosed over 2 h to remove the Boc protecting group. Upon completion of the addition, the suspension is stirred for ca. 5 h to ensure complete conversion to F8. Water is then added at 25° C. to dissolve the bis-hydrochloride salt of F8. The resulting biphasic mixture is stirred for ~2 h at 35° C. to ensure dissolution of the desired product. The layers are separated at JT 30° C.: the lower aqueous phase (containing product) is transferred to a tank while the upper organic phase (containing impurities from F7) is discarded. The aqueous phase is transferred into a new reactor via an in-line filter. An IPC of the aqueous layer is then taken to ensure the absence of F7. In case F7 is not completely converted, the temperature is raised to 40° C. for one hour before allowing the solution to cool down to RT. The resulting aqueous product-containing solution is then neutralized at 25° C. with sodium hydroxide (ca. 30% w/w) until a pH value of 5.0-5.5 is reached. Ethanol is then added to the resulting suspension and the temperature is increased to 60° C. Subsequently, 1 M aqueous sodium hydroxide solution is dosed until a pH value of 7.5-8.5 is reached. The product suspension is allowed to cool to 25° C. over 2 h and stirred further for ~1 h. The F8 crystals are isolated by filtration and the filter cake is washed with ethanol. The F8 wet product is dried at JT 50° C. under reduced pressure.

### Example 6: Preparation of F11

Starting material F8 is suspended in ethyl acetate. Sodium carbonate (1.2 equiv) is added to the suspension. The suspension is heated to 50° C. A solution of acrylic anhydride (F9, 1.05 equiv) in ethyl acetate is added to the suspension over at least 1 h. The reaction mixture is stirred for about 30 min at 50° C. After addition of water, the reaction mixture is stirred for about 30 min at 65° C. The phases are separated at 60° C. and the aqueous phase is removed. To the organic phase 0.05M sulfuric acid is added and stirred for ca. 15 min at 60° C. The aqueous phase is removed at 60° C. Afterwards, the organic phase is washed with water and the aqueous phase is removed 60° C. The final organic phase is treated by low in particles filtration at 65° C. At internal temperature of 60° C. a distillation is conducted at reduced pressure to remove ca. 25% the solvent mixture, while simultaneously adding ethyl acetate to keep the solvent level about constant. Thereby the water content is reduced. To the solution a seed suspension of a crystalline form (anhydrous modification A as disclosed in WO2020/234779) in ethyl acetate is added. The suspension is stirred for at least 15 min. A second distillation with internal temperature of 60° C. is conducted at reduced pressure to remove ca. 12% of the solvent mixture, while simultaneously adding ethyl acetate to keep the solvent level about constant. The suspension is cooled down to 30° C. in 200 min. At internal temperature of 30° C. a third distillation is conducted at reduced pressure, while simultaneously adding ethyl acetate to keep the solvent level about constant. The suspension is cooled down to 0° C. within 200 min and left to stir for at least 240 min at 0° C. The product is isolated by centrifugation and the filter cake is washed twice with ethyl acetate. The isolated wet product is dried on trays in a drying oven at 40° C. under vacuum. As product F11 is obtained.

### Example 7—Genotoxicity of 5-fluoro-2-methyl-3-(4,4,5,5-tetramethyl-1,3,2-dioxaborolan-2-yl)aniline (“INT 3” from WO 2015/079417)

Compound INT 3 is a key intermediate in the synthesis of remibrutinib described in Example 6 of WO 2015/079417. INT 3 was therefore subjected to AMES testing (bacterial reverse mutation test) to determine whether there were any genotoxicity-related safety issues. Under the testing conditions used and applying standard mutagenicity criteria, INT 3 was found to have mutagenic potential in the tester strain TA97a in the presence of metabolic activation.

The objective of the Salmonella/microsome assay is to evaluate the mutagenic potential of a test item by its effects on one or more histidine-requiring strains of Salmonella typhimurium in the absence and presence of a liver-metabolising system. The Ames assay is a rapid, reliable and economical method for screening compounds for potential genetic activity at the nucleotide level. A large database has been accumulated with this assay, confirming its ability to detect genetically active compounds of most chemical classes with around 80 to 90% sensitivity and specificity.

With the exception of strain TA102, these strains require biotin as well as histidine for growth. In strain TA102 the critical mutation in the histidine gene is located on a multicopy plasmid pAQ1. This strain is particularly sensitive to the activities of oxidative and cross-linking mutagens. The plasmid derivatives (TA98, TA100, TA97a and TA102) have increased sensitivity to certain mutagens as the pKM101 plasmid codes for an error-prone DNA repair system^{(1,3)}.

When exposed to a mutagen, some of the bacteria in the treated population, through chemical interaction with the compound, undergo genetic changes which cause them to revert to a non-histidine-requiring state and thus grow in the absence of exogenous histidine. Different tester strains are used because each is mutated by particular chemical classes of compound. A compound that is mutagenic in one strain need not be so in another^{(1,3)}.

### Methods

Test item: INT 3, also known as 5-fluoro-2-methyl-3-(4,4,5,5-tetramethyl-1,3,2-dioxaborolan-2-yl)aniline. Vehicle: Di-methyl sulfoxide (DMSO) Purity/Content of drug: 97.95%. Molecular Structure:

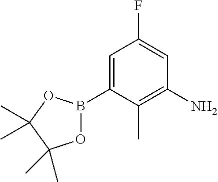 [SMILES: Cc1c(N)cc(F)cc1B1OC(C)(C)C(C)(C)O1]

Strains of Salmonella typhimurium used^{(4,5,6)}: TA98, TA100, TA1535, TA97a and TA102. Metabolic activation system⁽²⁾: Liver S-9 mix from male rats, Aroclor 1254-pretreated. Per plate, 0.5 mL of 5% S-9 mix was added. Controls: Control treatments comprised additions at the same volume per plate (0.1 mL) as the test item solutions. Negative controls comprised treatments with the chosen vehicle. The positive control chemicals were supplied and used as shown in the following table:

| | Stock** | Final | | |
|---|---|---|---|---|
| | concentration | concentration | | Use |
| Chemical* | (μg/mL) | (μg/plate) | Strains(s) | S-9 |
| 2-nitrofluorene | 20 | 2 | TA98 | − |
| (2NF) | | | | |
| Sodium | 10 | 1 | TA100, TA1535 | − |
| azide(NaN3) | | | | |
| Mitomycin C | 5 | 0.5 | TA 102 | − |
| (MMC) | | | | |
| 2- | 40 | 4 | TA98, TA100, | + |
| aminoanthracene | | | TA1535, TA97a | |
| (AAN) | 300 | 30 | TA102 | + |

*Obtained from Sigma-Aldrich.
**Stock solutions were formulated in DMSO.

### Results

Concentrations tested (Mutaqenicity Study): 50, 158, 501, 1582 and 5000 μg/plate (using all strains+/−S-9). Precipitation and toxicity: In the preliminary cytotoxicity as well as in mutagenicity test, the test item did not indicate any cytotoxicity in any of the strain, both in the presence and absence of metabolic activation. Also the test item did not precipitate up to highest concentration, both in the presence and absence of metabolic activation. Mutagenicity: Data from control treatments confirmed the correct strain and assay functioning, and the data were accepted as valid.

Following Experiment 1 treatment with INT3, increases in the number of revertants which exceeded 2-fold the concurrent vehicle controls (2.3 fold) were observed in strain TA97a at 5000 μg/plate, in the presence of metabolic activation. In order to further assess these increases in revertant numbers, a further experiment was conducted in strain TA97a in the presence and absence of metabolic activation.

Following Experiment 2 treatments with INT3, no doubling in the revertant number over the concurrent vehicle controls (which is formally the criterion for a positive response) was observed in strain TA97a at 5000 μg/plate, in presence of metabolic activation. However, at the highest test concentration a 1.8 fold increase was obtained. Increases of 2.3-fold (exceeding the 2-fold threshold indicating mutagenic potential of the test item) and 1.8 fold in the two independent experiments indicated that the test item, INT3, was considered to have weak mutagenic potential in strain TA97a in presence of metabolic activation.

No other increases in revertant numbers of at least 2-fold (1.5-fold for strain TA102) the concurrent vehicle controls were observed following any other strain treatments.

Acceptance criteria: The assay was considered valid as all the following criteria were met:
1. Vehicle control counts fell within the normal ranges;
2. The positive control chemicals induced increases in revertant numbers by 5- to 30-fold for different strains when compared to the concurrent vehicle controls confirming discrimination between different strains, and an active S-9 preparation.

Evaluation criteria: For valid data, the test item was considered mutagenic in this assay if a concentration related increase in revertant numbers of 2-fold (in strains TA98, TA100, TA1535 or TA97a) or 1.5-fold (in strain TA102) the concurrent vehicle control values was observed. The test item was regarded positive in this assay if the above criterion was met. The test item was regarded negative in this assay if the above criterion was not met.

### REFERENCES (FOR EXAMPLE 7)

1) Bruce N. Ames, Joyce Mccann and Edith Yamasaki, 1975. Methods for detecting carcinogens and mutagens with Salmonella/Mammalian-Microsome mutagenicity test. Mut. Res., 31:347-364.
2) Bruce N. Ames, William E. Durston, Edith Yamasaki and Frank D. Lee, 1973, Carcinogens are mutagens: A simple test system combining liver homogenates for activation and bacteria for detection. Proc. Nat. Acad. Sci. USA., 70 No. 8: 2281-2285.
3) Dorothy M. Maron and Bruce N. Ames, 1983, Revised methods for the Salmonella mutagenicity test. Mut. Res., 113:173-215.
4) ICH Harmonised Tripartite Guideline Guidance; S2 (R1), “On Genotoxicity Testing and Data Interpretation for Pharmaceuticals Intended for Human Use”; At Step 4 of the Process the final draft is recommended for adoption to the regulatory bodies Current Step 4 version, dated 9 Nov. 2011.
5) Lutz Müller et. al., 1999, ICH-Harmonised guidances on genotoxicity testing of pharmaceuticals: evolution, reasoning and impact. Mut. Res., 436:195-225.
6) OECD Guidelines for the Testing of Chemicals; No. 471; “Bacterial Reverse Mutation Test”; Adopted 21 Jul. 1997.

1. A synthesis method comprising converting compound X6b and compound F6 into compound F7: 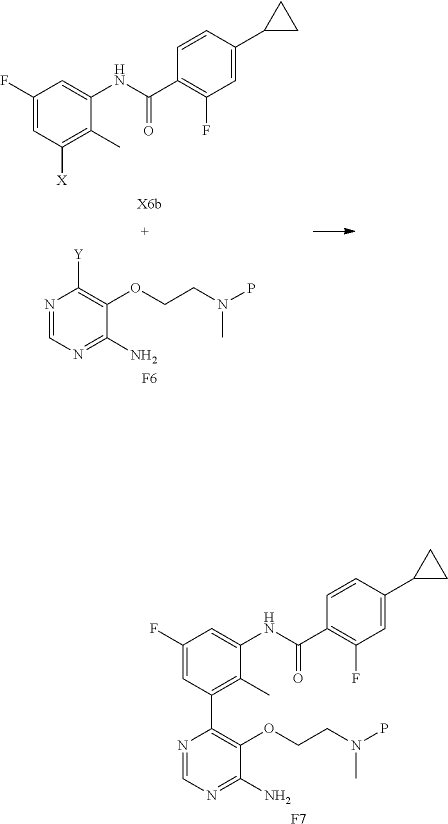
wherein X and Y are each independently Cl, Br, or I, and wherein P is an amine protecting group.

2. The method according to claim 1, wherein P is a carbamate protecting group or an acetamide protecting group or a sulfonamide protecting group.

3. The method of claim 1 further comprising the borylation of X6b to give X6a: 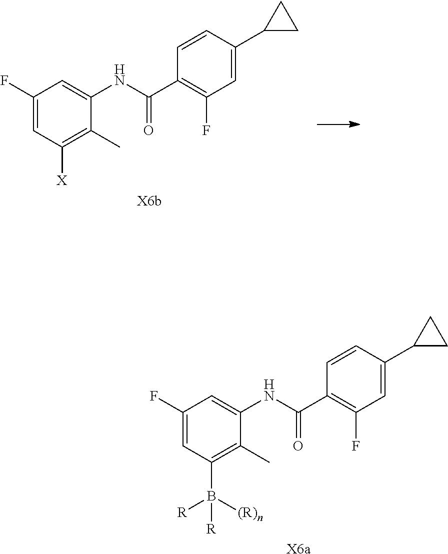
wherein X is F, Cl, Br, or I, n is 0 or 1 and R is F, Cl, Br, or I, OH, OC₁-C₆ alkyl, N(C₁-C₆ alkyl)₂, aryl, or wherein two or three R groups other than F, Cl, Br, I, or OH can be taken together to form a cyclic boronate ester, for example pinacol boronate, or N-methyliminodiacetic acid (MIDA) boronate.

4. (canceled)

5. The method according to claim 3, wherein the borylation is conducted using one or more catalysts, one or more ligands, one or more borylating agents, and/or one or more bases, and optionally one or more additives.

6. The method according to claim 5, wherein the borylating agent is selected from the group consisting of diboron compounds, boronic acids, and organoborates.

7. The method according to claim 6, wherein the borylating agent is selected from the group consisting of bis(pinacolato)diboron, bis(catecholato)diborane, B₂(NMe₂)₄, bis-boronic acid, mono-, di-, or tri-C₁-C₆alkylborate, mono-, di-, or tri-methylborate, mono-, di-, or tri-ethylborate, mono-, di-, or tri-propylborate, mono-, di-, and of tri-propenylate acid.

8. The method according to claim 5, wherein the metal catalyst contains palladium, nickel, or copper, or a combination thereof.

9. The method according to claim 8, wherein the metal catalyst is provided as a pre-catalyst complex PdCl₂(PtBuPh₂)₂ or a Buchwald pre-catalyst selected from: 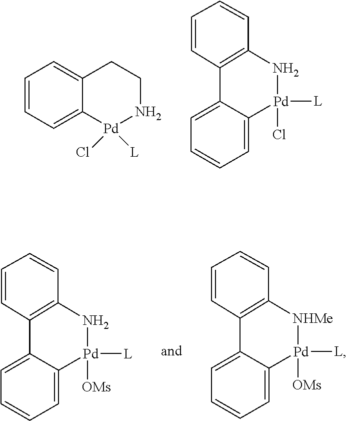
complexed to a phosphine ligand L.

10. The method according to claim 8, wherein the metal catalyst is provided as a pre-catalyst, selected from Pd(MeCN)₂Cl₂, Pd(TFA)₂, and PdBr₂, with the ligand t-BuPPh₂.

11. The method according to claim 5, wherein the ligand is selected from the group consisting of an organophosphine, an N-heterocyclic carbene, a diazabutadiene, a dibenzylideneacetone, and combinations thereof.

12. The method according to claim 11, wherein the ligand is an organophosphine ligand, for example an organophosphine selected from the group consisting of XPhos, APhos, CPhos, RuPhos, SPhos, cataCXium, DavePhos, JohnPhos, MePhos, XantPhos, Cy₃P—HBF₄, SPhos-SO₃Na, Cy-BIPHEP, t-BuPPh₂ and PPh₃ and combinations thereof.

13. The method of claim 12, wherein the ligand:catalyst molar ratio is from 1:1 to 3:1.

14. The method according to claim 5, wherein the base is an inorganic salt selected from KOH, NaOH, Ca(OH)₂, Na₂CO₃, K₂CO₃, Cs₂CO₃, KOAc, NaOAc, diisopropylethylamine (DIPEA), and triethylamine, or a combination thereof.

15. The method according to claim 5, wherein the additive is present and is an alcohol.

16-19. (canceled)

20. The method according to claim 5 wherein the borylation step is characterized by at least one of the following:
i. the catalyst is Pd(MeCN)₂Cl₂ in an amount of 0.1 mol % to 2 mol % relative to the number of moles of X6b, 0.1 mol % to 1.5 mol %, or 0.25 mol % or 0.5 mol % relative to the number of moles of X6b;
ii. the ligand is tBuPPh₂ in an amount of 0.2 mol % to 4% relative to the number of moles of X6b, or 0.2 mol % to 3 mol %, or 0.5 mol % or 1 mol % relative to the number of moles of X6b;
iii. the catalyst is Pd(MeCN)₂Cl₂, the ligand is tBuPPh₂ and the number of moles of tBuPPh₂ is twice or three times the number of moles of Pd(MeCN)₂Cl₂;
iv. the borylating agent is bis(pinacolato)diboron in an amount of 1 to 2 molar equivalents relative to X6b, about 1.05 molar equivalents relative to X6b;
v. the base is KOAc in an amount of 2 to 5 molar equivalents relative to X6b, or 2.5 equivalents relative to X6b;
vi. the temperature of the borylation step is 30° C. to 120° C., or 40° C. to 50° C., or 60° C. or 70° C.

21. The method of claim 20 wherein the borylation step is:

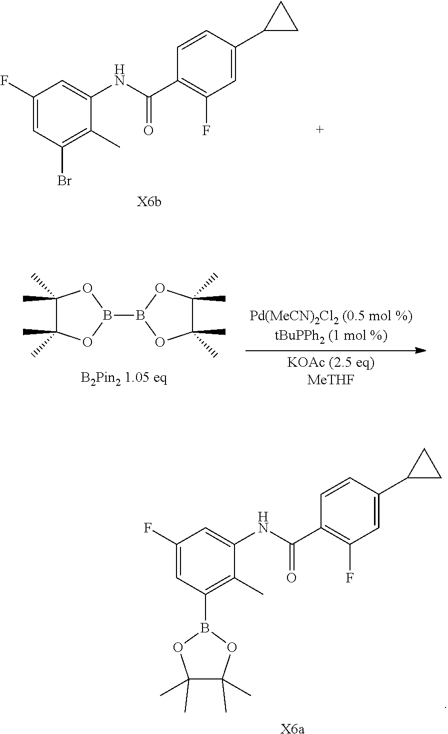

22. The method according to claim 3, wherein X6a and F6 are converted to via Suzuki coupling, wherein the Suzuki coupling is conducted using one or more catalysts, one or more ligands, and/or one or more bases, and optionally one or more additives.

23. The method according to claim 22, wherein the catalyst is a metal catalyst containing palladium, nickel, or copper, or a combination thereof.

24. The method according to claim 23, wherein the ligand is selected from the group consisting of an organophosphine, an N-heterocyclic carbene, a diazabutadiene, a dibenzylideneacetone, and combinations thereof.

25. The method according to claim 24, wherein the ligand is an organophosphine ligand selected from the group consisting of XPhos, APhos, CPhos, RuPhos, SPhos, Sphos-SO3Na, cataCXium, DavePhos, JohnPhos, MePhos, XantPhos, t-BuPPh₂, PPh₃ and combinations thereof.

26. (canceled)

27. The method according to claim 23, wherein the metal catalyst is provided as a pre-catalyst (e.g. Pd(MeCN)₂Ph₂) together with a ligand (e.g. t-BuPPh₂).

28. The method according to claim 22, wherein the coupling is conducted in alcoholic solvent, ether-based solvent, aqueous solvent or mixture thereof.

29. The method according to claim 22, wherein the coupling is characterized by at least one of the following:
vii. The catalyst or pre-catalyst is present in an amount of 0.1 mol % to 5 mol %, or 0.25 mol % to 3 mol %, or 0.5 mol % to 1.5 mol %, or 0.5 mol % or 1 mol % relative to the number of moles of F6 or X6a;
viii. The number of moles of ligand, if present, is twice or 3 times the number of moles of catalyst or pre-catalyst;
ix. The molar ratio of F6:X6a is from 2:1 to 1:2, or from 1.5:1 to 1:1.5, 1.2:1 to 1:1.2 or 1:1;
x. the additive is optional and when present is in an amount of 2 to 5 molar equivalents relative to F6 or X6a; and/or
xi. The base is in an amount of 2 to 5 molar equivalents, or in an amount of 2 to 3 molar equivalents, or in an amount of 3 molar equivalent relative to the number of moles of F6 or X6a;

30-31. (canceled)

32. The method according to claim 22, wherein the coupling is characterized by at least one of the following
i) the catalyst is Pd(MeCN)₂Cl₂ in an amount of 0.25 mol % to 2 mol % relative to the number of moles of X6b, 0.25 mol % to 1.5 mol %, 0.5 mol % or 1 mol % relative to the number of moles of X6b;
ii) the ligand is tBuPPh₂ in an amount of 0.5 mol % to 4% relative to the number of moles of X6b, 1 mol % or 2 mol % relative to the number of moles of X6b; and the number of moles of tBuPPh₂ is twice the number of moles of Pd(MeCN)₂Cl₂;
iii) the base is KOH in an amount of 2 to 5 molar equivalents, or 3 molar equivalents relative to X6b;
iv) the coupling is conducted in MeTHF and water mixture; and
i) the temperature of the coupling is 30° C. to 70° C.

33. The method according to claim 32, wherein the coupling is:

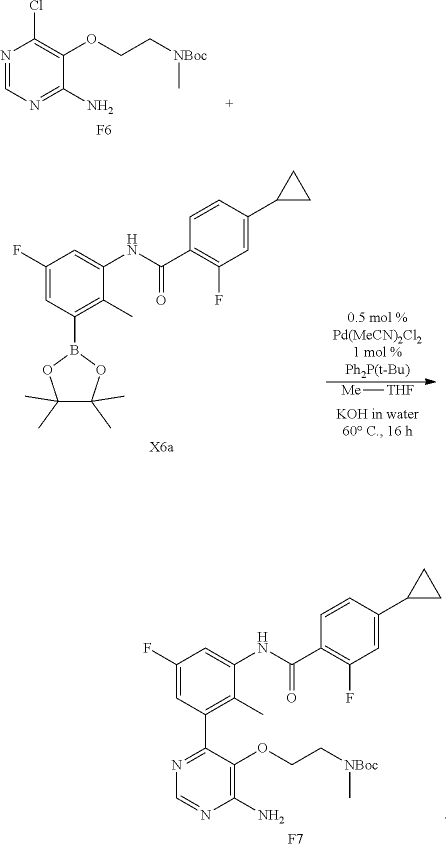

34. (canceled)

35. The method according to claim 32, wherein the borylation and coupling are conducted in a one-pot synthesis.

36. The method according to claim 35, wherein the conversion of X6b and F6 to F7 is:

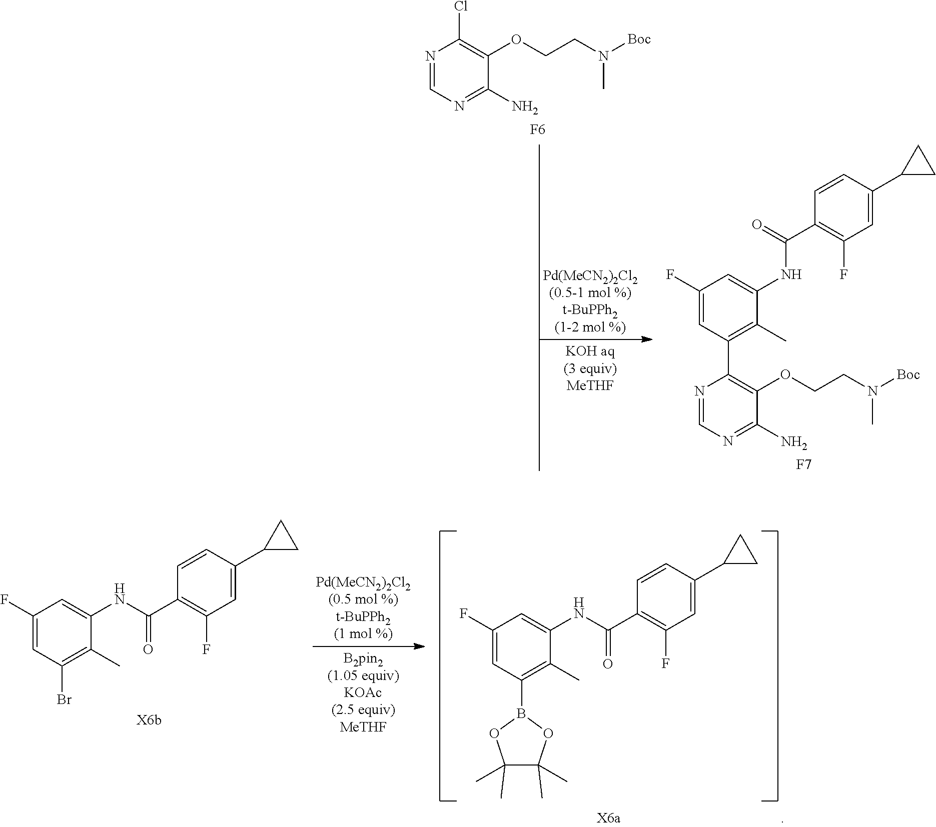

37. The method according to claim 36, wherein the the conversion of X6b and F6 to F7 is conducted in polar organic solvent, selected from an ether solvent and an alcoholic solvent.

38. A method for making a synthetic intermediate X6b: 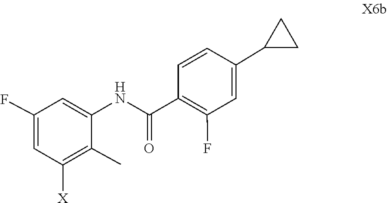
wherein X is F, Cl, Br, or I; the method comprising reacting compound X6d with compound N6a: 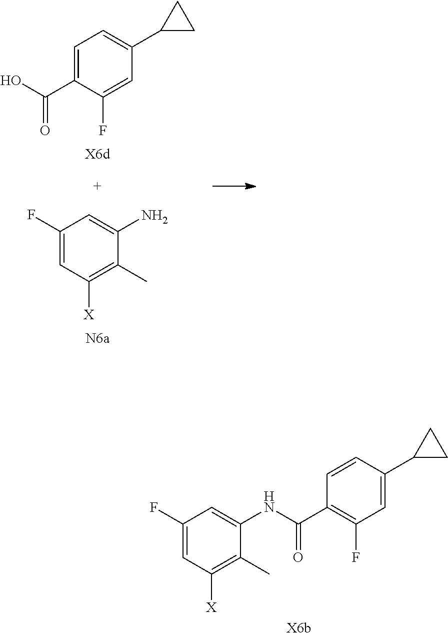
wherein X is Cl, Br, or I.

39-69. (canceled)

70. The method of any preceding claim, wherein the method is used in the synthesis of compound F11:

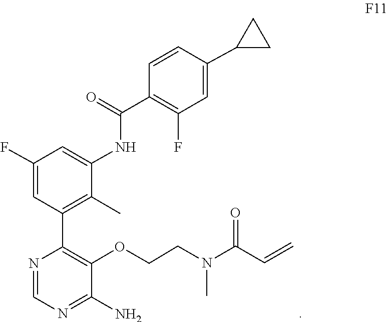 [SMILES: C=CC(=O)N(C)CCOc1c(N)ncnc1-c1cc(F)cc(NC(=O)c2ccc(C3CC3)cc2F)c1C]

71-74. (canceled)

75. A synthetic intermediate X6b: 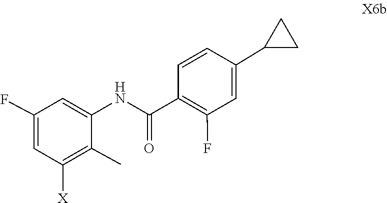
wherein X is Cl, Br, or I.

76: The method according to claim 20 wherein the borylation step is characterized by at least one of the following:
xii. the catalyst is Pd(MeCN)₂Cl₂ in an amount of 0.5 mol % relative to the number of moles of X6b;
xiii. the ligand is tBuPPh₂ in an amount of 1 mol % relative to the number of moles of X6b;
xiv. the catalyst is Pd(MeCN)₂Cl₂, the ligand is tBuPPh₂ and the number of moles of tBuPPh₂ is twice the number of moles of Pd(MeCN)₂Cl₂,
xv. the borylating agent is bis(pinacolato)diboron in an amount of about 1.05 molar equivalents relative to X6b;
xvi. the base is KOAc in an amount of 2.5 equivalents relative to X6b;
xvii. the temperature of the borylation step is 60° C. or 70° C.

77. The method of claim 76 wherein the borylation step is:

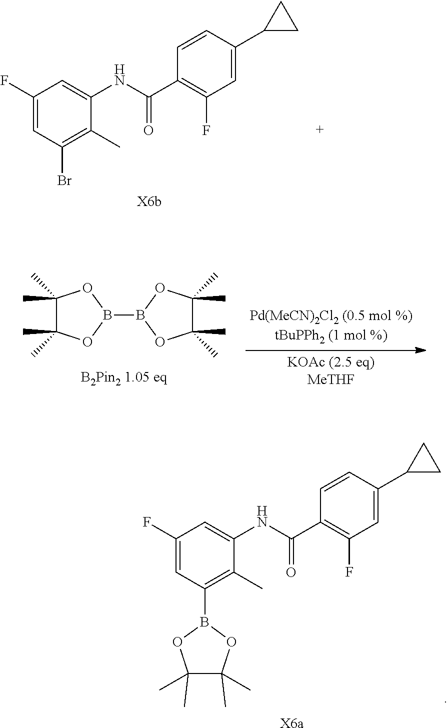

78. The method according to claim 77, wherein X6a and F6 are converted to F7 via Suzuki coupling, wherein the Suzuki coupling is conducted using one or more catalysts, one or more ligands, and/or one or more bases, and optionally one or more additives.

79. The method according to claim 78, wherein the coupling is characterized by at least one of the following
i) the catalyst is Pd(MeCN)₂Cl₂ in an amount of 0.5 mol % or 1 mol % relative to the number of moles of X6b;
ii) the ligand is tBuPPh₂ in an amount of 1 mol % or 2 mol % relative to the number of moles of X6b; and the number of moles of tBuPPh₂ is twice the number of moles of Pd(MeCN)₂Cl₂;
iii) the base is KOH in an amount of 3 molar equivalents relative to X6b;
iv) the coupling is conducted in MeTHF and water mixture; and
ii) the temperature of the coupling is 60° C.

80. The method according to claim 79, wherein the coupling is:

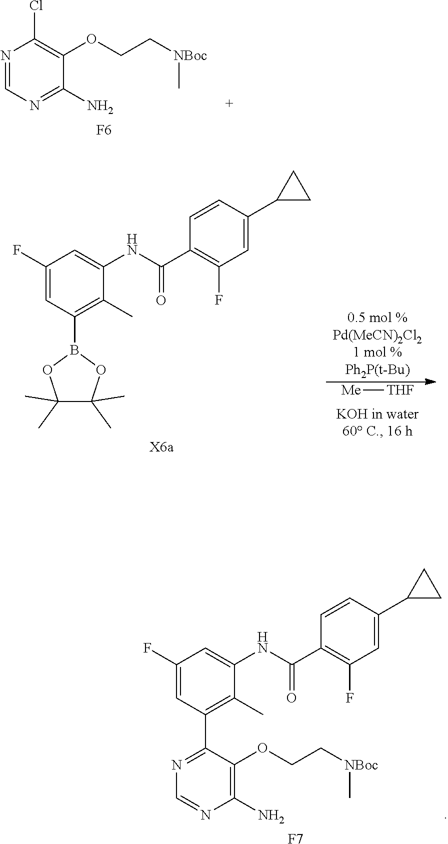

81. The method according to claim 79, wherein the borylation and coupling are conducted in a one-pot synthesis.
